(19) United States
(12) Patent Application Publication
(10) Pub. No.: US 2013/0260422 A1
(43) Pub. Date: Oct. 3, 2013

(54) Compositions and Methods Relating to Variant DNA Polymerases and Synthetic DNA Polymerases

(71) Applicants: Jennifer Ong, Salem, MA (US); Thomas C. Evans, Topsfield, MA (US); Nathan Tanner, Peabody, MA (US)

(72) Inventors: Jennifer Ong, Salem, MA (US); Thomas C. Evans, Topsfield, MA (US); Nathan Tanner, Peabody, MA (US)

(73) Assignee: NEW ENGLAND BIOLABS, INC., Ipswich, MA (US)

(21) Appl. No.: 13/823,811

(22) PCT Filed: Aug. 31, 2012
(86) PCT No.: PCT/US12/53330

§ 371 Date: Mar. 15, 2013

Related U.S. Application Data
(60) Provisional application No. 61/605,484, filed on Mar. 1, 2012.
(60) Provisional application No. 61/530,273, filed on Sep. 1, 2011.

(51) Int. Cl. C12N 9/12

(52) U.S. Cl. CPC C12N 9/1276
USPC 435/91.2; 435/194; 536/23.2; 435/91.51; 435/252.33

(57) ABSTRACT

Compositions of novel polymerase variants and methods of identifying, making and using these novel polymerases are described. The variants have been shown to have advantageous properties such as increased thermostability, deoxyuridine nucleoside triphosphate tolerance, salt tolerance, reaction speed and/or increased reverse transcriptase properties. Uses for these improved enzymes have been demonstrated in isothermal amplification such as LAMP. Enhanced performance resulting from the use of these variants in amplification has been demonstrated both in reaction vessels and in dedicated automated amplification platforms.

### FIELD OF THE INVENTION

A DNA polymerase from Geobacillus stearothermophilus has been described in Kong, et al., U.S. Pat. No. 5,814,506 (1998). This enzyme, which is a Bst DNA polymerase, belongs to DNA polymerase Family A and shares about 45% sequence identity with its better known relative Taq DNA polymerase. Whereas Taq DNA polymerase is from a hyperthermophilic organism and is able to survive the high temperatures of the polymerase chain reaction, the Bst DNA polymerase reported in Kong, et al., is from a thermophilic organism, is optimally active between 60-70° C., but does not survive the high temperatures of PCR. The full length (FL) Bst DNA polymerase is 876 amino acid residues and has 5′-3′ endonuclease activity but not 3′-5′ exonuclease activity. The large fragment (LF) of Bst DNA polymerase lacks both 5′-3′ exonuclease activity and 3′-5′ exonuclease activity and is only 587 amino acid residues with 289 amino acids being deleted from the N-terminal end. The FL Bst DNA polymerase and the LF Bst DNA polymerase have been found to be useful for isothermal amplification techniques and DNA sequencing.

### SUMMARY OF EMBODIMENTS OF THE INVENTION

Compositions and methods are described herein that relate to variants of DNA polymerases belonging to Family A DNA polymerases.

In embodiment 1, a variant Family A DNA polymerase comprises two or more amino acid sequence motifs selected from 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569, where the number preceding the amino acid in the motif corresponds to the location of that amino acid in the amino acid sequence of FIG. 1, wherein the two or more motifs confer improved reaction speed in an amplification reaction and/or improved stability compared to the reaction speed and/or stability of any of SEQ ID NOs:1-23.

Other embodiments are defined in claims 2-49 appended hereto.

In embodiment 2, a variant polymerase of embodiment 1 has at least 75% but less than 100% identity to any of SEQ ID NOs:1-23.

In embodiment 3, a variant polymerase of embodiment 1 or 2 comprises at least three or four or five or six or seven or eight or nine or ten or eleven or twelve of the motifs.

In embodiment 4, a variant polymerase of any one of the preceding embodiments further comprises one or more mutations selected from the group of mutations consisting of (a)-(f) where the mutations in (a)-(f) are:
(a) A1E, G3(K, E or D), K5(L, A or V), E8(M or A), E9D, M10I, A13(D, E, T or V), I14D, V15A, V17(T, E or G), I18V;
(b) E20(M or D) M34(Q or L), E36D, I46F, L48(N or I), M57(L or I), P59(T or A), T61L, D65S, S66(F, E or P);
(c) Q67A, L69(V or K), A73E, M81V, A84R, V88(A or I), R99V, A102D, N113A, D117(T, S or A), A118D, G119(D or E), I121(A or V), V124K, E131H, 5135(E or P), V144A, 5147(P or A), L148(D or V), Q152(L or P);
(d) T153(A or V), Q170(R or E), M173(L or I), D175E, N178(E, K or R), Q183(L, E or R), L185F, T186(L or I), K187(E or D), Q190(L or M);
(e) A193(I or S), A194(L, S or T), N205(D or K), S216(L or E), R223(V, K or G), A224E, I225(Q or V), V247L, R307H, M316R; and
(f) A330T, D357L, D378N, D380E, I383A, Q387R, L390M; 1400V, E406D, A410S, N411R, A433S, N437G, T439K, A452E, Q459(R or E), N463(V, E or D), L484D, D486E, V494L, T501M, Q530R, I552M, E557(K, Q or R) and T568(E or R).

In embodiment 5, a variant polymerase according to embodiment 4, comprising at least one mutated amino acid selected from each of groups (a)-(f).

In embodiment 6, a variant polymerase of embodiment 4 further comprises two, three, four, five, six, seven, eight, nine, ten, eleven, twelve, thirteen, fourteen, fifteen, sixteen, seventeen, eighteen, nineteen, or twenty mutant amino acids at the same position as the corresponding amino acids in (a)-(f) of SEQ ID NO:1.

In embodiment 7, a variant polymerase of any one of the preceding embodiments is described wherein said sequence motif(s) confer one or more improved properties selected from at least one of specific activity; reaction speed; thermostability; storage stability; dUTP tolerance and salt tolerance; increased performance in isothermal amplification; non-interference of pH during sequencing; improved strand displacement; altered processivity; altered ribonucleotide incorporation; altered modified nucleotide incorporation; and altered fidelity when compared to the corresponding parent polymerase.

In embodiment 8, a variant polymerase of any one of the preceding embodiments is described wherein a peptide is fused to one end of the variant polymerase directly or by means of a linker sequence.

In embodiment 9, an enzyme preparation comprises a variant polymerase according to any one of the preceding embodiments and a buffer.

In embodiment 10, an enzyme preparation according to embodiment 8 or 9 comprises a temperature dependent inhibitor of polymerase activity.

In embodiment 11, an enzyme preparation according to any of embodiments 8 through 10, further comprises dNTPs.

In embodiment 12, a DNA encodes a variant polymerase as described in any of preceding embodiments.

In embodiment 13, a host cell comprises the DNA according to embodiment 12.

In embodiment 14, a process for preparing a variant of a parent Family A DNA polymerase having improved polymerase activity compared with the parent polymerase, comprises synthesizing a polypeptide as defined in any one of embodiments 1-8; and characterizing the polymerase activity.

In embodiment 15, the process of embodiment 14 is described wherein characterizing the polymerase activity, further comprises: determining in comparison with the parent polymerase, at last one of: thermostability; stability in storage; tolerance to salt; performance in isothermal amplification; strand displacement; kinetics; processivity; fidelity; altered ribonucleotide incorporation; altered dUTP incorporation; and altered modified nucleotide incorporation.

In embodiment 16, a variant Family A DNA polymerase is obtainable by the process of embodiment 14 or embodiment 15.

In embodiment 17, a variant polymerase of any of embodiments 1 through 7 wherein the one or more motifs or one or more mutations selected from the group of mutations consisting of (a)-(f) have improved reverse transcriptase (Rtx) activity.

In embodiment 18, a method for reverse transcribing an RNA of interest, comprises combining an RNA with a DNA polymerase variant or preparation thereof according to embodiments 1-11 to form a complementary DNA (cDNA).

In embodiment 19, a method according to embodiment 18 further comprises amplifying the cDNA by means of the DNA polymerase variant or preparation thereof according to claims 1-11, to produce amplified DNA.

In embodiment 20, a method for amplifying DNA comprises combining a target DNA with a DNA polymerase variant or preparation thereof according to embodiments 1-11, to produce amplified DNA.

In embodiment 21, a variant protein comprises: an amino acid sequence with at least 75% or 80% or 85% or 90% or 95% but less than 100% sequence identity to any of SEQ ID NOs:1-23, wherein the variant protein further comprises at least one mutated amino acid having a position corresponding to SEQ ID NO:1 selected from the group of mutated amino acids consisting of (a)-(f) where the mutations in (a)-(f) are:
(a) A1E, G3(K, E or D), K5(L, A or V), E8(M or A), E9D, M10I, A13(D, E, T or V), I14D, V15A, V17(T, E or G), I18V;
(b) E20(M or D) M34(Q or L), E36D, I46F, L48(N or I), M57(L or I), P59(T or A), T61L, D65S, S66(F, E or P);
(c) Q67A, L69(V or K), A73E, M81V, A84R, V88(A or I), R99V, A102D, N113A, D117(T, S or A), A118D, G119(D or E), I121(A or V), V124K, E131H, 5135(E or P), V144A, 5147(P or A), L148(D or V), Q152(L or P);
(d) T153(A or V), Q170(R or E), M173(L or I), D175E, N178(E, K or R), Q183(L, E or R), L185F, T186(L or I), K187(E or D), Q190(L or M);
(e) A193(I or S), A194(L, S or T), N205(D or K), 5216(L or E), R223(V, K or G), A224E, I225(Q or V), V247L, R307H, M316R; and
(f) A330T, D357L, D378N, D380E, I383A, Q387R, L390M; 1400V, E406D, A410S, N411R, A433S, N437G, T439K, A452E, Q459(R or E), N463(V, E or D), L484D, D486E, V494L, T501M, Q530R, I552M, E557(K, Q or R) and T568(E or R).

In embodiment 22, the variant may contain at least one amino acid corresponding to a mutated amino acid in SEQ ID NO:1 selected from each of groups (a) through (f).

In embodiment 23, the variant protein according to embodiment 21, further comprises at least one amino acid motif or at least two amino acid motifs selected from the group consisting of: from 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569.

In embodiment 24, a variant protein according to any of embodiments 21-23, further comprises two, three, four, five, six, seven, eight, nine, ten, eleven, twelve, thirteen, fourteen, fifteen, sixteen, seventeen, eighteen, nineteen, or twenty amino acids at the same positions as the corresponding mutant amino acids in (a)-(f) of SEQ ID NO:1.

In embodiment 25, the variant protein of embodiment 24 is described wherein the amino acid sequence is at least 80% identical to any one of SEQ ID NOs:1-23.

In embodiment 26, a variant protein according to embodiment 25 is described, wherein the amino acid sequence is at least 90% identical to any one of SEQ ID NOs:1-23.

In embodiment 27, a variant protein according to embodiment 26, is described wherein the amino acid sequence is at least 95% identical to any one of SEQ ID NOs:1-23.

In embodiment 28, a non-naturally occurring synthetic protein comprises: a fragment 1, a fragment 2, a fragment 3, a fragment 4, a fragment 5, a fragment 6, a fragment 7 and a fragment 8 wherein the fragments are covalently linked in numerical order, and wherein:

the fragment 1 is selected from Segment 1 having an amino acid sequence selected from the group consisting of SEQ ID NOs:24-39;

the fragment 2 is selected from Segment 2 having an amino acid sequence selected from the group consisting of SEQ ID NOs:40-56;

the fragment 3 is selected from Segment 3 having an amino acid sequence selected from the group consisting of SEQ ID NOs:57-72;

the fragment 4 is selected from Segment 4 having an amino acid sequence selected from the group consisting of SEQ ID NOs:73-87;

the fragment 5 selected from Segment 5 having an amino acid sequence selected from the group consisting of SEQ ID NOs: 88-99;

the fragment 6 selected from Segment 6 having an amino acid sequence selected from the group consisting of SEQ ID NOs:100-111;

the fragment 7 selected from Segment 7 having an amino acid sequence selected from the group consisting of SEQ ID NOs:112-125;

the fragment 8 selected from Segment 8 having an amino acid sequence selected from the group consisting of SEQ ID NOs:126-138; and;

wherein the covalently linked fragments has an amino acid sequence that does not have 100% identity to SEQ ID NOs:1-23.

In embodiment 29, a synthetic protein according to embodiment 28 is described, wherein the amino acid sequence of the synthetic protein comprises at least one amino acid sequence motif selected from the group consisting of: 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569.

In embodiment 30, the synthetic protein of embodiment 28 is described, wherein the amino acid sequence comprises at least two or three or four or five or six or seven or eight or nine or ten or eleven or twelve of the amino acid sequence motifs selected from the group consisting of: 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569.

In embodiment 31, a protein comprises at least 75% or 80% or 85% or 90% or 95% sequence identity with SEQ ID NOs:1 and further comprises one or more mutations (such as, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 mutations) selected from the group consisting of A1E, G3(K, E or D), K5(L, A or V), E8(M or A), E9D, M10I, A13(D, E, T or V), I14D, V15A, V17(T, E or G), I18V, E20(M or D) M34(Q or L), E36D, I46F, L48(N or I), M57(L or I), P59(T or A), T61L, D65S, S66(F, E or P), Q67A, L69(V or K), A73E, M81V, A84R, V88(A or I), R99V, A102D, N113A, D117(T, S or A), A118D, G119(D or E), I121(A or V), V124K, E131H, 5135(E or P), V144A, 5147(P or A), L148(D or V), Q152(L or P), T153(A or V), Q170(R or E), M173(L or I), D175E, N178(E, K or R), Q183(L, E or R), L185F, T186(L or I), K187(E or D), Q190(L or M): A193(I or S), A194(L, S or T), N205(D or K), 5216(L or E), R223(V, K or G), A224E, I225(Q or V), V247L, R307H, M316R, A330T, D357L, D378N, D380E, I383A, Q387R, L390M, 1400V, E406D, A410S, N411R, A433S, N437G, T439K, A452E, Q459(R or E), N463(V, E or D), L484D, D486E, V494L, T501M, Q530R, I552M, E557(K, Q or R) and T568(E or R); and optionally a sequence motif at a specified position in SEQ ID NO:1 selected from the group consisting of: 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569.

In embodiment 32, a variant protein or a synthetic protein according to any of embodiments of 21-31 is described, wherein a peptide is fused to one end of the variant protein. For example, the peptide may be fused to one end of the variant protein either directly or by means of a linker.

In embodiment 33, an enzyme preparation comprises a variant protein or a synthetic protein according to any of embodiments 21-31 and a buffer.

In embodiment 34, an enzyme preparation according to embodiment 33 further comprises a plurality of proteins.

In embodiment 35, an enzyme preparation according to embodiment 33 or 34 further comprises a reversible inhibitor of polymerase activity.

In embodiment 36, an enzyme preparation according to embodiment 33 or 34 further comprises dNTPs.

In embodiment 37, DNA encodes a variant protein or synthetic protein described in any of embodiments 21-36.

In embodiment 38, a host cell comprises the DNA according to embodiment 37.

In embodiment 39, a method for obtaining a variant of a parent protein has improved polymerase activity compared with the parent protein, comprises synthesizing a protein from any of embodiments 21-36; and characterizing the polymerase activity.

In embodiment 40, which is a method according to embodiment 39, characterizing the polymerase activity further comprises: determining in comparison with the parent protein, at least one of: thermostability; stability in storage; tolerance to salt; performance in isothermal amplification; strand displacement; kinetics; processivity; fidelity; altered ribonucleotide incorporation; altered dUTP incorporation; and altered modified nucleotide incorporation. Additionally, characterizing the polymerase activity includes detecting an increase in Rtx activity.

In embodiment 41, a method comprises:
(a) synthesizing a protein wherein the protein has an amino acid sequence which is capable of being generated from single selected protein fragments obtainable from 8 different segments described in FIG. 2; and
(b) assaying the synthetic protein for polymerase activity.

In embodiment 42, a method according to embodiment 41 is provided, wherein the protein is synthesized by cloning a DNA sequence encoding the protein.

In embodiment 43, a method comprises:
(a) selecting a protein variant or synthetic protein according to any of claims embodiments 21-36 having an amino acid sequence; and
(b) expressing the protein variant or synthetic protein as a fusion protein with an additional peptide at an end of the amino acid sequence.

In embodiment 44, a method of isothermal amplification comprises:
(a) providing a preparation comprising a variant protein or synthetic protein according to any of claims 21-36;
(b) combining a target DNA with the preparation; and
(c) amplifying the target DNA at a temperature less than 90° C.

In embodiment 45, a method according to embodiment 44 is described, wherein the amplification reaction results in a quantitative measure of the amount of target DNA in the preparation.

In embodiment 46, a DNA polymerase having one or more improved properties for isothermal amplification compared with SEQ ID NO:1, where the one or more improved properties are selected from the group consisting of:
(a) an increased reaction speed where the increase is at least 10% and as much as 200%; 500% or 1000%;
(b) an increased temperature stability in the range of 50° C. to 100° C., 50° C. to 90° C., or 60° C. to 90° C.;
(c) an increased salt tolerance in the range of 10 mM-1 M, or 20 mM-200 mM or 500 mM monovalent salt;
(d) an increase in storage stability at 25° C., retaining at least 50% activity over 45 weeks, over 1 year or over 2 years;
(e) an enhanced dUTP tolerance of the range of an increase of 50% to 100% dUTP; and
(f) an increased reverse transcriptase activity by at least 2 fold; wherein the DNA polymerase is a non-naturally occurring mutant of a wild type Bst DNA polymerase.

In embodiment 47, a DNA polymerase according to embodiment 46 is described having at least two or three or four or five or six of the improved properties.

In embodiment 48, a DNA polymerase according to embodiments 46 or 47 having at least 80% amino acid sequence identity but less than 100% amino acid sequence identity with any of SEQ ID NOs:1-23 and containing at least 12 artificially introduced single amino acid mutations that occur within a three amino acid motif that differs from a three amino acid motif in the corresponding site of a naturally occurring Bst polymerase.

In embodiment 49, a DNA polymerase according to embodiment 48 is described wherein at least one of the three amino acid motifs is selected from the group consisting of 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569.

In general in one aspect, the composition includes a variant protein, having an amino acid sequence with at least 75% or 80% or 85% or 90% or 95% but less than 100% identity to any of SEQ ID NOs:1-23. The variant protein may include at least one amino acid identified by a position in its amino acid sequence and an identity corresponding to any of the mutated amino acids in the corresponding position in SEQ ID NO:1 and listed in (a)-(f) as provided below, wherein the at least one amino acid is selected from the group consisting of:
(a) A1E, G3(K, E or D), K5(L, A or V), E8(M or A), E9D, M10I, A13(D, E, T or V), I14D, V15A, V17(T, E or G), I18V;
(b) E20(M or D) M34(Q or L), E36D, I46F, L48(N or I), M57(L or I), P59(T or A), T61L, D65S, S66(F, E or P);
(c) Q67A, L69(V or K), A73E, M81V, A84R, V88(A or I), R99V, A102D, N113A, D117(T, S or A), A118D, G119(D or E), I121(A or V), V124K, E131H, 5135(E or P), V144A, 5147(P or A), L148(D or V), Q152(L or P);
(d) T153(A or V), Q170(R or E), M173(L or I), D175E, N178(E, K or R), Q183(L, E or R), L185F, T186(L or I), K187(E or D), Q190(L or M);
(e) A193(I or S), A194(L, S or T), N205(D or K), 5216(L or E), R223(V, K or G), A224E, I225(Q or V), V247L, R307H, M316R; and
(f) A330T, D357L, D378N, D380E, I383A, Q387R, L390M; 1400V, E406D, A410S, N411R, A433S, N437G, T439K, A452E, Q459(R or E), N463(V, E or D), L484D, D486E, V494L, T501M, Q530R, I552M, E557(K, Q or R) and T568(E or R).

In another aspect, the variant may contain at least one amino acid corresponding to a mutated amino acid in SEQ ID NO:1 and selected from each of groups (a) through (f).

In another aspect, the variant protein may include in addition to the amino acids specified above, two, three, four, five, six, seven, eight, nine, ten, eleven, twelve, thirteen, fourteen, fifteen, sixteen, seventeen, eighteen, nineteen, or twenty amino acids at the same positions and identities as the corresponding mutant amino acids in (a)-(f) of SEQ ID NO:1.

In another aspect, the variant protein may include at least one or two or three or four or five or six or seven or eight or nine or ten or eleven or twelve amino acid sequence motifs selected from 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569, where the number preceding the amino acid in the motif corresponds to the location of that amino acid in the amino acid sequence as determined from FIG. 1. The variant protein may include at least one or two or three or four or five or six or seven or eight or nine or ten or eleven or twelve of these motifs in addition to one or more mutations in (a)-(f).

In another aspect, the variant protein has an amino acid sequence that is at least 80%, or at least 85% or at least 90% or at least 95% but less than 100% identical to any one of SEQ ID NOs:1-23.

In another aspect, the variant protein of the sort described above has an amino acid sequence that is at least 80%, or at least 85% or at least 90% or at least 95% but less than 100% identical to any one of SEQ ID NOs:1-23.

In another aspect, a DNA polymerase is provided that comprises or consists of a plurality of peptide fragments selected from segments 1-8 covalently linked to form a single polypeptide that has less than 100% amino acid sequence identity with any of SEQ ID NOs:1-23.

In another aspect, a non-naturally occurring synthetic protein is provided that includes 8 fragments wherein the fragments include a Fragment 1 selected from Segment 1 and having an amino acid sequence selected from the group consisting of SEQ ID NOs:24-39; a Fragment 2 selected from Segment 2 and having an amino acid sequence selected from the group consisting of SEQ ID NOs:40-56, a Fragment 3 selected from Segment 3 and having an amino acid sequence selected from the group consisting of SEQ ID NOs:57-72, a Fragment 4 selected from Segment 4 and having an amino acid sequence selected from the group consisting of SEQ ID NOs:73-87, a Fragment 5 selected from Segment 5 and having an amino acid sequence selected from the group consisting of SEQ ID NOs:88-99; a Fragment 6 selected from Segment 6 and having an amino acid sequence selected from the group consisting of SEQ ID NOs:100-111; a Fragment 7 selected from Segment 7 and having an amino acid sequence selected from the group consisting of SEQ ID NOs:112-125; and a Fragment 8 selected from Segment 8 and having an amino acid sequence selected from the group consisting of SEQ ID NOs:126-138. Fragments 1-8 are covalently linked preferably in numerical order so as to form a single protein wherein the single protein is not any of SEQ ID NOs:1-23.

In another aspect, the amino acid sequence of the synthetic protein comprises at least one or at least two or at least three or at least four or at least five or at least six or at least seven or at least eight or at least nine or at least ten or at least eleven amino acid sequence motifs selected from the group consisting of: 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569.

In another aspect, a non-naturally occurring protein is provided that comprises or consists of an amino acid sequence having at least 80% sequence identity with SEQ ID NO:1. The non-naturally occurring protein further comprises one or more mutations (such as, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 mutations) selected from the group consisting of A1E, G3(K, E or D), K5(L, A or V), E8(M or A), E9D, M10I, A13(D, E, T or V), I14D, V15A, V17(T, E or G), I18V, E20(M or D) M34(Q or L), E36D, I46F, L48(N or I), M57(L or I), P59(T or A), T61L, D65S, S66(F, E or P), Q67A, L69(V or K), A73E, M81V, A84R, V88(A or I), R99V, A102D, N113A, D117(T, S or A), A118D, G119(D or E), I121(A or V), V124K, E131H, 5135(E or P), V144A, 5147(P or A), L148(D or V), Q152(L or P), T153(A or V), Q170(R or E), M173(L or I), D175E, N178(E, K or R), Q183(L, E or R), L185F, T186(L or I), K187(E or D), Q190(L or M), A193(I or S), A194(L, S or T), N205(D or K), 5216(L or E), R223(V, K or G), A224E, I225(Q or V), V247L, R307H, M316R, A330T, D357L, D378N, D380E, I383A, Q387R, L390M, 1400V, E406D, A410S, N411R, A433S, N437G, T439K, A452E, Q459(R or E), N463(V, E or D), L484D, D486E, V494L, T501M, Q530R, I552M, E557(K, Q or R) and T568(E or R); and optionally a sequence motif at a specified position in SEQ ID NO:1 selected from the group consisting of: 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569.

In another aspect, a variant or synthetic protein as described herein may additionally comprise a peptide fused to the N-terminal end or the C-terminal end of the protein directly or via a linker.

In another aspect of the embodiments, an enzyme preparation is provided which contains a variant protein or a synthetic protein as described above and a buffer. The enzyme preparation may additionally contain a plurality of proteins described herein and/or a reversible inhibitor of polymerase activity and/or dNTPs.

In another aspect of the embodiments, a polynucleotide is provided that encodes a variant protein or synthetic protein as described above. The polynucleotide may be expressed in a transformed host organism.

In general, methods are provided for synthesizing a variant or synthetic protein of the type described above having polymerase activity which in one aspect includes synthesizing a protein of the sort described above; and optionally determining whether the protein has a desired property associated with polymerase activity, the polymerase activity being selected from the group consisting of increased thermostability; stability in storage; improved tolerance to salt; increased performance in isothermal amplification; does not alter the pH of a solution during sequencing; improved strand displacement; improved kinetics; altered processivity; altered ribonucleotide incorporation, altered non-standard deoxyribonucleotide incorporation; altered dUTP incorporation; higher fidelity; and increased Rtx activity as compared with the protein of any of SEQ ID NOs:1-23.

In another aspect, the method includes (a) synthesizing a protein wherein the protein has an amino acid sequence which is capable of being generated from single selected protein fragments obtainable from 8 different segments described in FIG. 2; and (b) assaying the synthetic protein for polymerase activity and properties associated therewith. The protein may be synthesized by cloning a DNA sequence encoding the protein.

In another aspect, a method is provided that includes selecting a protein variant or synthetic DNA polymerase protein from those described above; and expressing the protein as a fusion protein with an additional peptide at one or both ends of the DNA polymerase amino acid sequence.

In another aspect, a method is provided for isothermal amplification that includes: (a) providing a preparation comprising of a variant protein or synthetic protein selected from those described above; (b) combining a target DNA with the preparation; and (c) amplifying the target DNA at a temperature less than 90° C. to obtain an amplified target and optionally obtaining a quantitative measure of the amount of amplified DNA in the preparation.

In another aspect there is provided a DNA polymerase having one or more improved properties for isothermal amplification compared with SEQ ID NO:1, wherein the improved properties are selected from the group consisting of:
(a) an increased reaction speed in the range where the increase is at least 10% and as much as 20%; 500% or 1000%;
(b) an increased temperature stability in the range of 50° C. to 100° C., 50° C. to 90° C. or 60° C. to 90° C.;
(c) an increased salt tolerance in the range of 10 mM-1 M, or 20 mM-200 mM or 500 mM monovalent salt;
(d) an increased storage stability at 25° C., retaining at least 50% activity over 45 weeks, over 1 year or over 2 years;
(e) an enhanced dUTP tolerance of the range of an increase of 50% to 100% dUTP; and
(f) an increased reverse transcriptase activity by at least 2 fold.

In the aforementioned aspect the DNA polymerase: (a) may have at least two or three or four or five or six of the improved properties; (b) may have at least 80% amino acid sequence identity but less than 100% amino acid sequence identity with any of SEQ ID NOs:1-23 and containing at least 12 artificially introduced single amino acid mutations that occur in a three amino acid motif that differs from an amino acid in the corresponding site of a naturally occurring Bst polymerase; or (c) may be such that at least one of the three amino acid motifs is selected from the group consisting of 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569.

### BRIEF DESCRIPTION OF THE FIGURES

FIG. 1A shows an alignment of 23 wild type Bst DNA polymerase (LF) sequences. Not shown is a methionine optionally added at the N-terminal end of each of SEQ ID NOs:1-23 to facilitate expression of the polymerase in a host cell.

FIG. 1B shows sequence pair distances of the sequences in FIG. 1A using the software program Lasergene MegAlign™ (DNASTAR, Madison, Wis.).

FIG. 2 shows a 115 fragments arrayed in 8 segments where a fragment selected from each segment joined in order to the neighboring fragment forms an intact synthetic protein having DNA reagent properties.

FIGS. 3A and 3B show melt peaks for a parent Bst DNA polymerase FL or LF and variant DNA polymerases.

FIG. 3A shows the melt peaks for the variant DNA polymerase (FL) which has a melting temperature (Tm)=73.5° C. (Δ) and the parent Bst DNA polymerase (FL) has a Tm=68° C. (◯).

FIG. 3B shows the melt peaks for a parent Bst DNA polymerase LF (◯) which has a Tm=65° C. while the variant DNA polymerase (Δ) has a Tm=70° C.

The reactions were performed in 1× Detergent-free ThermoPol™ Buffer (New England Biolabs, Ipswich, Mass.) and 1× SYPRO Orange (Life Technologies, Carlsbad, Calif.).

FIGS. 4A-E show how the properties of a variant DNA polymerase can be screened for significant beneficial properties using an isothermal amplification protocol (Notomi, et al., Nucleic Acids Research, 28:E63 (2000)) and lambda DNA.

FIG. 4A shows an analysis of reaction speed. The variant DNA polymerase shows faster DNA amplification than the parent Bst DNA polymerase.

FIG. 4B shows the results of an assay to determine salt tolerance. The time in which the amplification reaction took to reach a threshold level of product was graphed against increasing KCl concentration in the reaction. The variant DNA polymerase was more tolerant to changes in salt concentration than the parent Bst DNA polymerase.

FIG. 4C shows the results of an assay to determine an increase in thermostability of a variant DNA polymerase by at least 3° C. compared with the parent Bst DNA polymerase. The time in which the amplification reaction took to reach a threshold level of product was graphed against increasing reaction temperature. The variant DNA polymerase was able to amplify DNA at a higher temperature than the parent Bst DNA polymerase.

FIG. 4D shows the results of an assay for storage stability in which a variant polymerase remains stable for at least 28 weeks at room temperature (22° C.) versus about 13 weeks for the parent Bst DNA polymerase (8000 U/ml for each enzyme was used).

FIG. 4E shows the results of an assay for dUTP tolerance in which a parent Bst DNA polymerase is significantly inhibited by increasing amounts of dUTP while the variant DNA polymerase activity is relatively stable as dUTP levels increase (1.4 mM dUTP corresponds to complete substitution of dTTP with dUTP). The ability to incorporate dUTP without inhibition of the polymerase is a useful feature of a DNA polymerase for various applications including strand modification and differentiation. Thermophilic archaeal DNA polymerases do not amplify DNA effectively in the presence of dUTP. Taq DNA polymerase can incorporate dUTP into substrate but Taq DNA polymerase is not suitable for isothermal amplification because it is not capable of the requisite amount of strand displacement.

FIGS. 5A and 5B shows that the DNA polymerase mutants described herein with improved polymerase activity also have improved reverse transcriptase activity.

FIG. 5A shows the results of determining Rtx activity using RT-qPCR. The lower the value of cycles (Cq) the greater the activity of the Rtx. From left to right, the bar chart shows Primer alone, RNA alone, Bst polymerase large fragment (BstLF), 2 mutants of the DNA polymerase described herein, Rtx, Avian Myeloblastosis Virus Reverse Transcriptase (AMV) and Moloney Murine Leukemia Virus Reverse Transcriptase (MMLV).

FIG. 5B shows gel electrophoresis of amplified DNA resulting from an RNA template and BstLF DNA polymerase or mutants. The lanes are labeled left to right as follows: primer alone, RNA alone, BstLF, Mutant 1 and 2, Rtx, AMV and MMLV.

### DETAILED DESCRIPTION OF THE EMBODIMENTS

As used herein, the term “synthetic” with respect to proteins or peptides refers to a non-naturally occurring amino acid sequence that is generated either by expression of a gene encoding the non-naturally occurring amino acid sequence or is generated by chemical synthesis. The gene encoding the non-naturally occurring amino acid sequence may be generated, for example, by mutagenesis of a naturally occurring gene sequence or by total chemical synthesis.

A “variant” protein refers to a protein that differs from a parent protein by at least one amino acid that is the product of a mutation. A variant polymerase is intended to include a “synthetic” protein and vice versa as the context permits. The examples utilize a variant DNA polymerase but it will be understood to a person of ordinary skill in the art that the assays described in the examples are applicable to analyzing synthetic proteins also.

“Non-naturally occurring” refers to a sequence or protein that at the date in which the embodiments of the invention are presented herein, no naturally occurring amino acid sequence corresponding to the alleged non-naturally occurring amino acid has been described in the publically available databases.

“Isothermal amplification” refers to a DNA amplification protocol that is conducted at a temperature below 90° C. after an initial denaturation step, where an initial denaturation step is required.

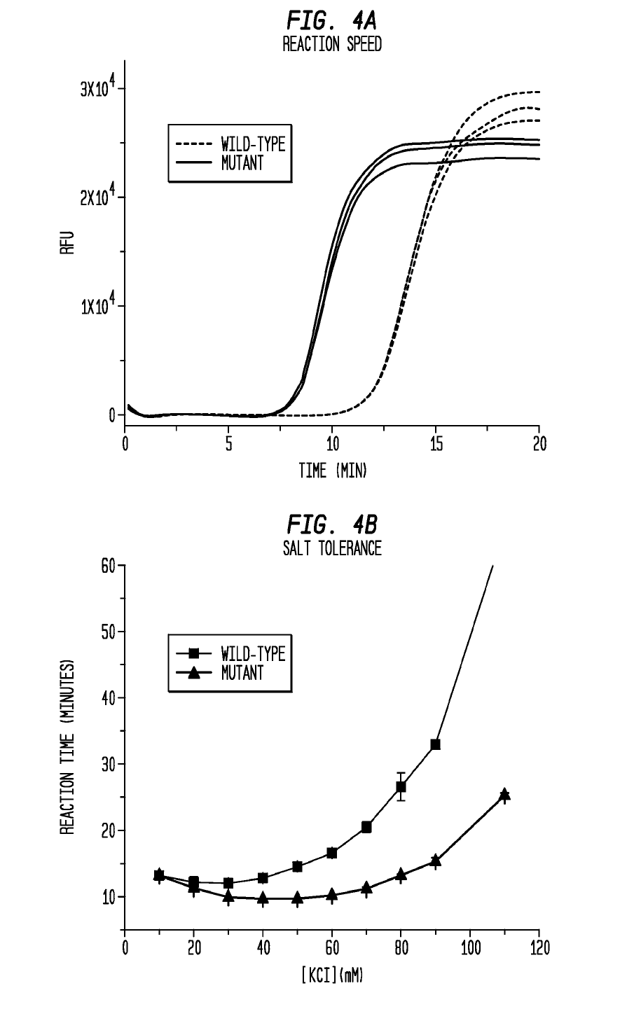

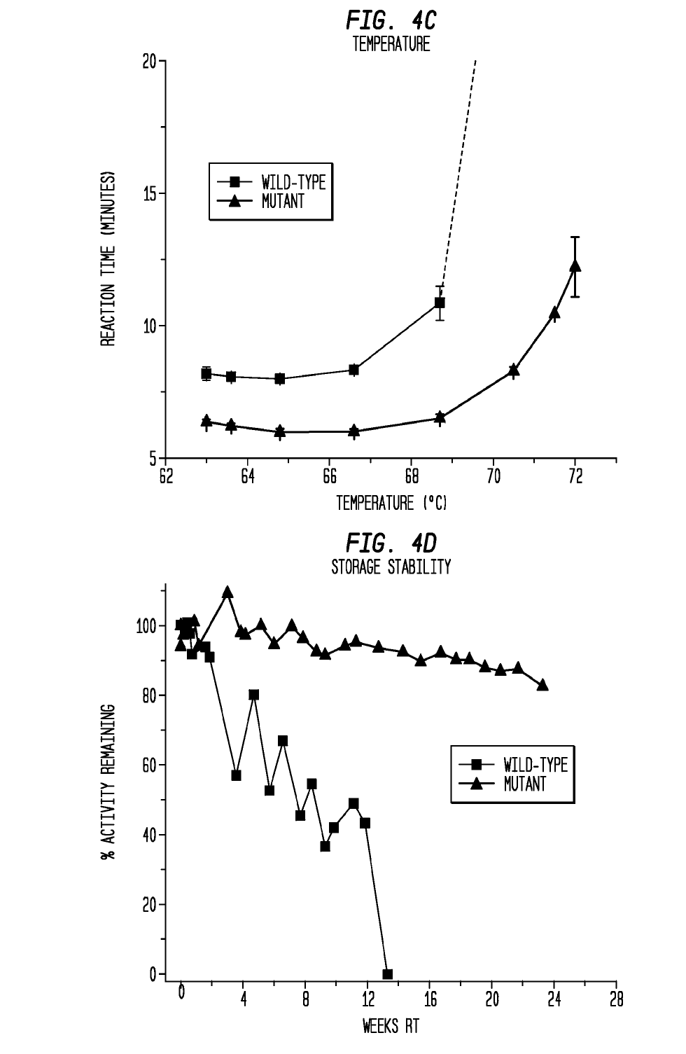

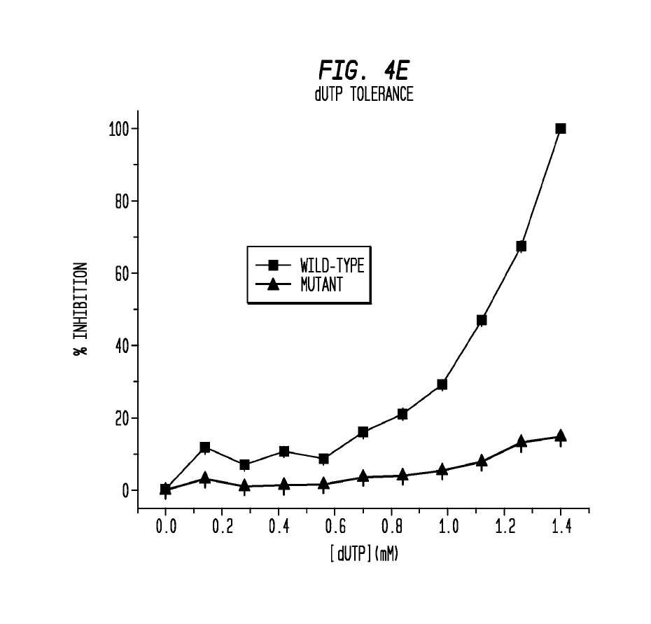

The term “stability” as used in the claims includes thermostability and storage stability as illustrated in FIG. 4 and in the examples.

We have developed a set of variant proteins that are mutants of a highly conserved family of DNA polymerases belonging to Family A DNA polymerases. One or more of the amino acid mutations and/or amino acid motifs described herein are capable of enhancing the properties of these polymerases such as those properties determined by the assays described in the examples.

The Family A DNA polymerases are highly conserved so that it will be readily appreciated that with the teaching of the present embodiments, a person of ordinary skill in the art could select a naturally occurring DNA polymerase sequence (such as from GenBank) having at least 80% sequence identity with SEQ ID NOs:1-23 and introduce one or more of the specified mutations and/or motifs described herein to obtain polymerases with improved properties such as the type described in the examples.

In one embodiment, the DNA polymerase mutant proteins comprise or consist of an amino acid sequence that has at least 75% amino acid sequence identity, at least 80% amino acid sequence identity, or at least 85% amino acid sequence identify and as much as 90% amino acid sequence identity or 95% amino acid sequence identity to the parent DNA polymerase provided in the sequences described in SEQ ID NOs:1-23 wherein the amino acid sequence is less than 100% identical to the amino acid sequence of any of SEQ ID NOs:1-23.

Percentage sequence identity may be calculated by any method known in the art such as for example, using the BLOSUM62 matrix and the methods described in Henikoff, et al., PNAS, 89 (22):10915-10919 (1992)).

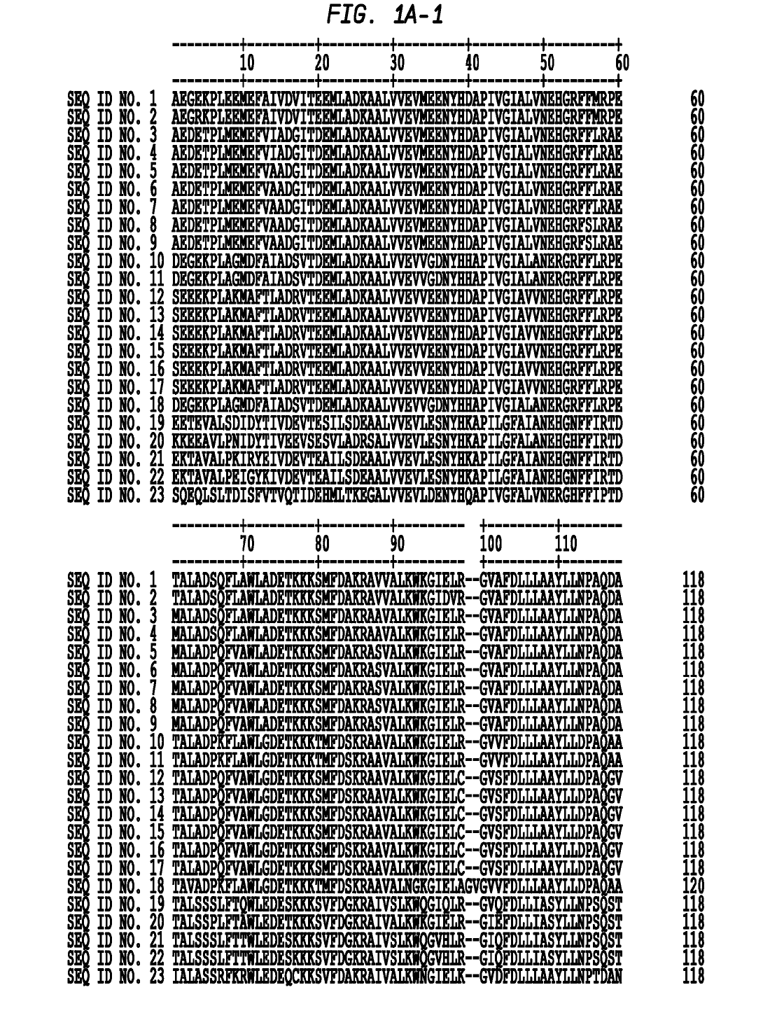

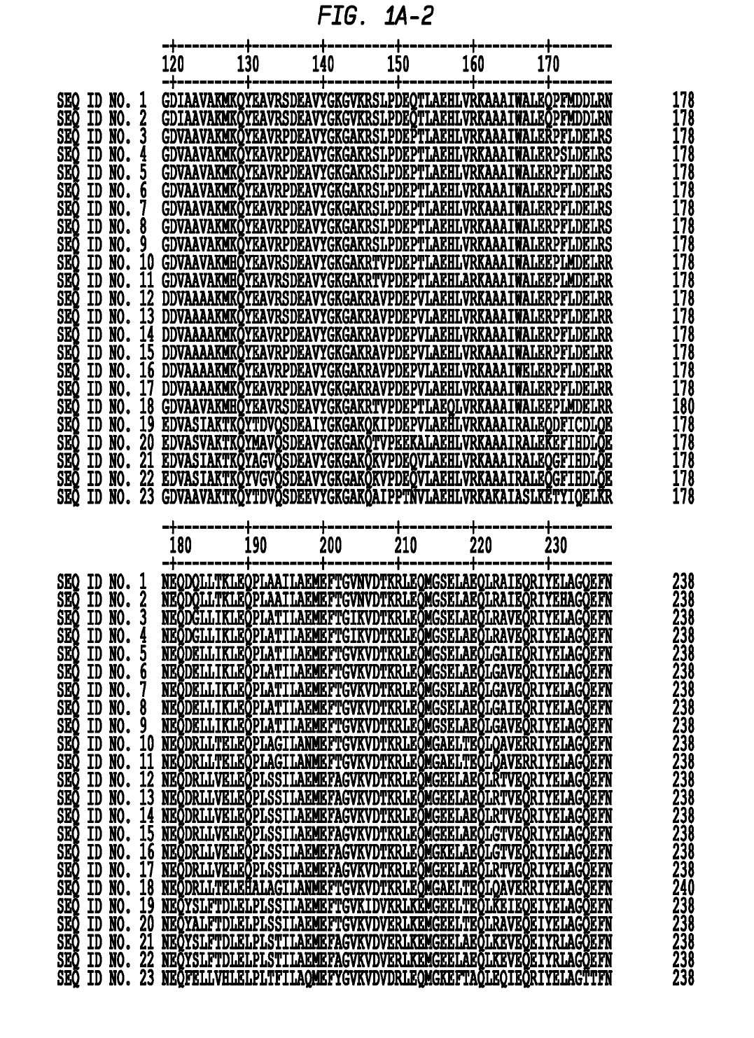

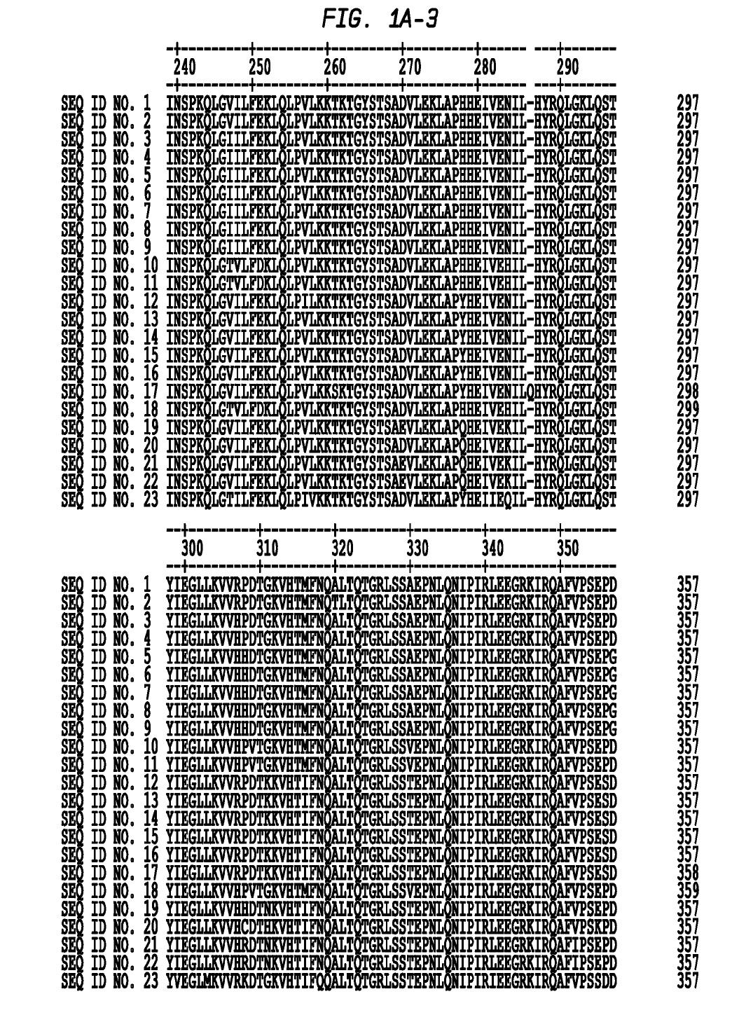

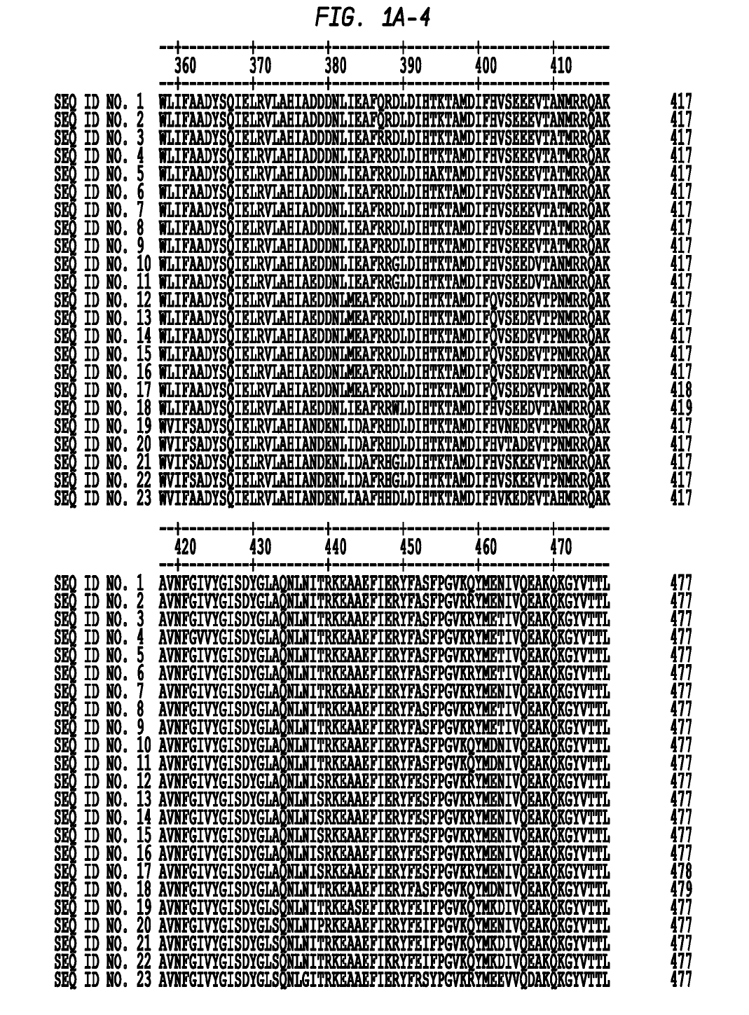

The at least one amino acid mutation in the variants is identified using the numbering scheme described in FIG. 1 with a reference amino acid as it occurs in SEQ ID NO:1 replaced by a desired amino at the specified position.

Accordingly, a parent polymerases having amino acid sequences with at least 75%, 80%, 85%, 90%, or 95% sequence identity to any of SEQ ID NOs:1-23 may be altered by at least one mutation selected from the group consisting of: A1E, G3(K, E or D), K5(L, A or V), E8(M or A), E9D, M10I, A13(D, E, T or V), I14D, V15A, V17(T, E or G), I18V, E20(M or D) M34(Q or L), E36D, I46F, L48(N or I), M57(L or I), P59(T or A), T61L, D65S, S66(F, E or P), Q67A, L69(V or K), A73E, M81V, A84R, V88(A or I), R99V, A102D, N113A, D117(T, S or A), A118D, G119(D or E), I121(A or V), V124K, E131H, S135(E or P), V144A, S147(P or A), L148(D or V), Q152(L or P), T153(A or V), Q170(R or E), M173(L or I), D175E, N178(E, K or R), Q183(L, E or R), L185F, T186(L or I), K187(E or D), Q190(L or M), A193(I or S), A194(L, S or T), N205(D or K), S216(L or E), R223(V, K or G), A224E, I225(Q or V), V247L, R307H, M316R, A330T, D357L, D378N, D380E, I383A, Q387R, L390M, 1400V, E406D, A410S, N411R, A433S, N437G, T439K, A452E, Q459(R or E), N463(V, E or D), L484D, D486E, V494L, T501M, Q530R, I552M, E557(K, Q or R) and T568(E or R).

The variant may optionally include one or more motifs selected from the group consisting of: 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569.

The DNA polymerase protein variants described above may be screened using at least one method described in Examples 1-6 so as to identify those variants having at least one of the functional properties that are at least typical of a Family A DNA polymerase, such as, Bst DNA Polymerase with an amino acid sequence corresponding to SEQ ID NO:1. The DNA polymerase may additionally have improved properties as compared with the wild type Family A DNA polymerases such as those including one of specific activity, reaction speed, thermostability, storage stability, dUTP tolerance, salt tolerance and reverse transcriptase activity.

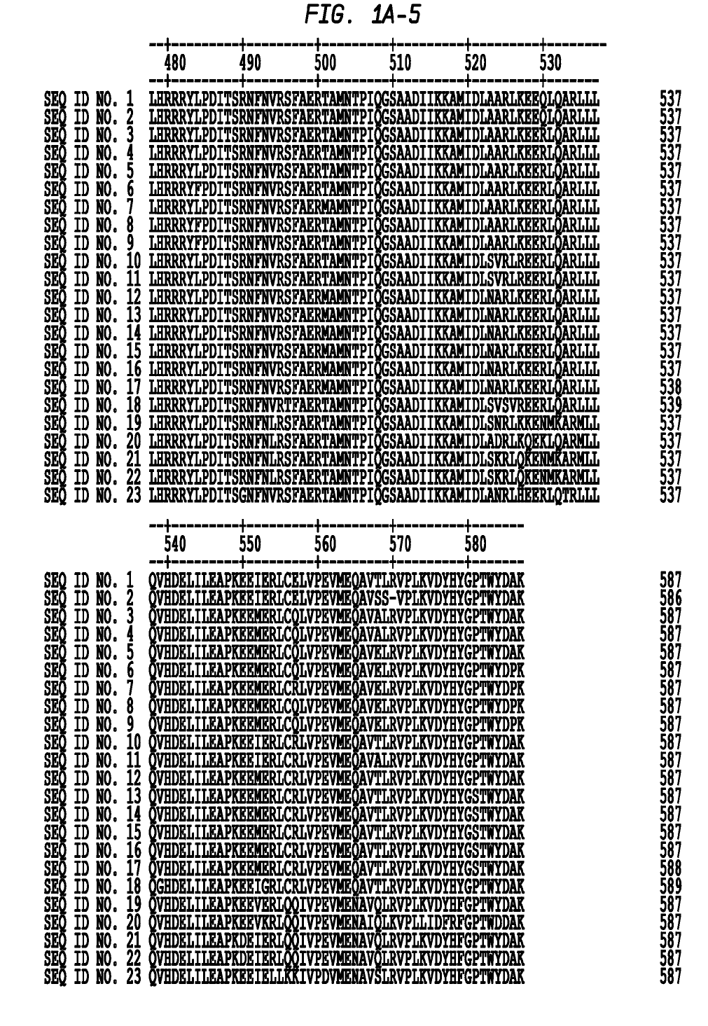

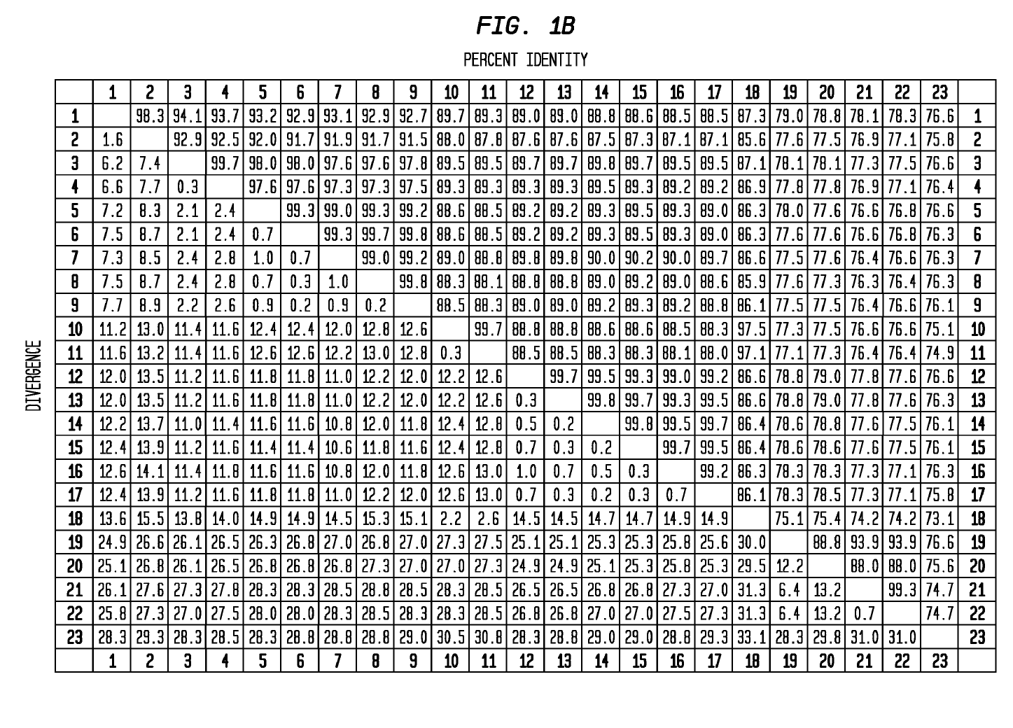

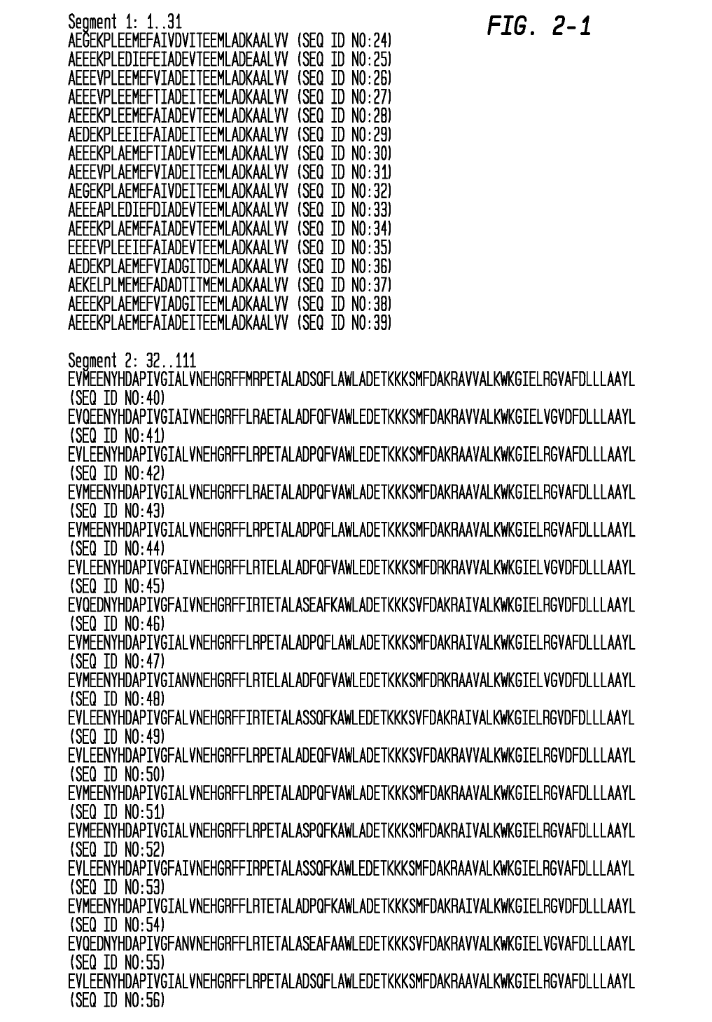

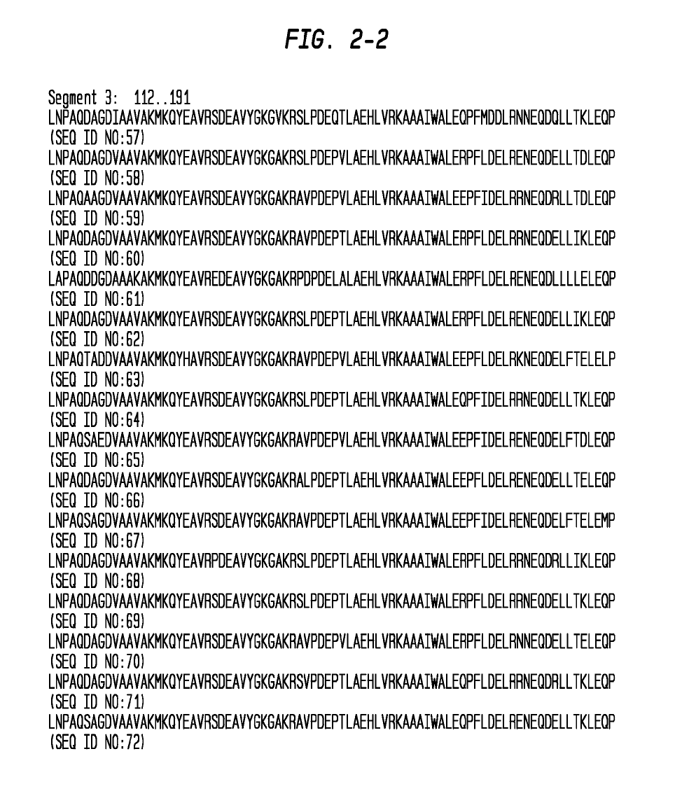

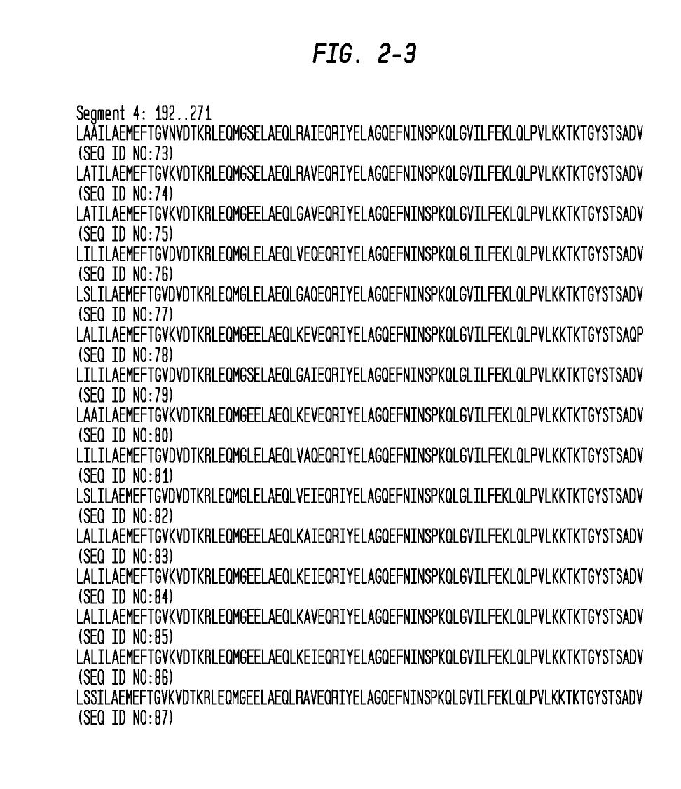

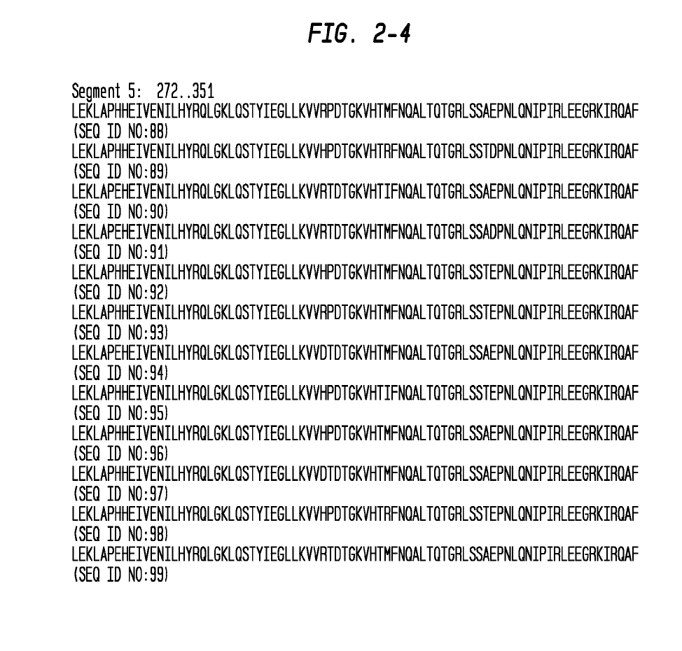

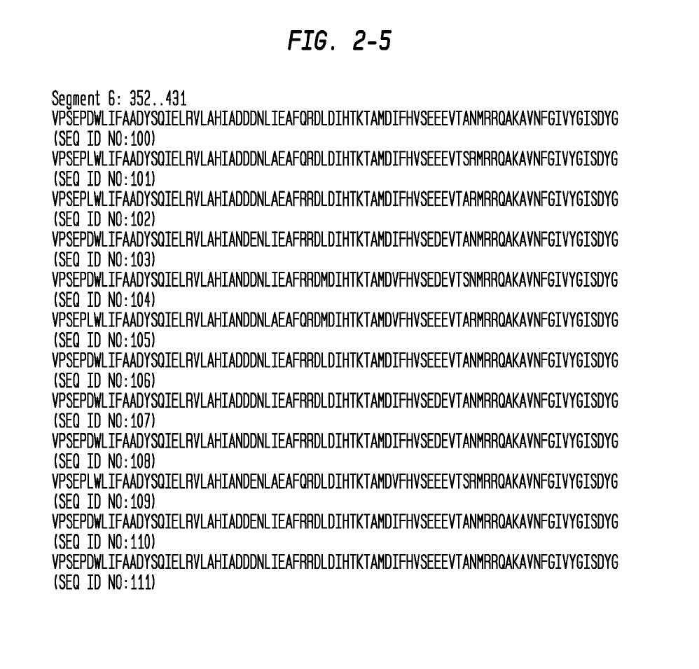

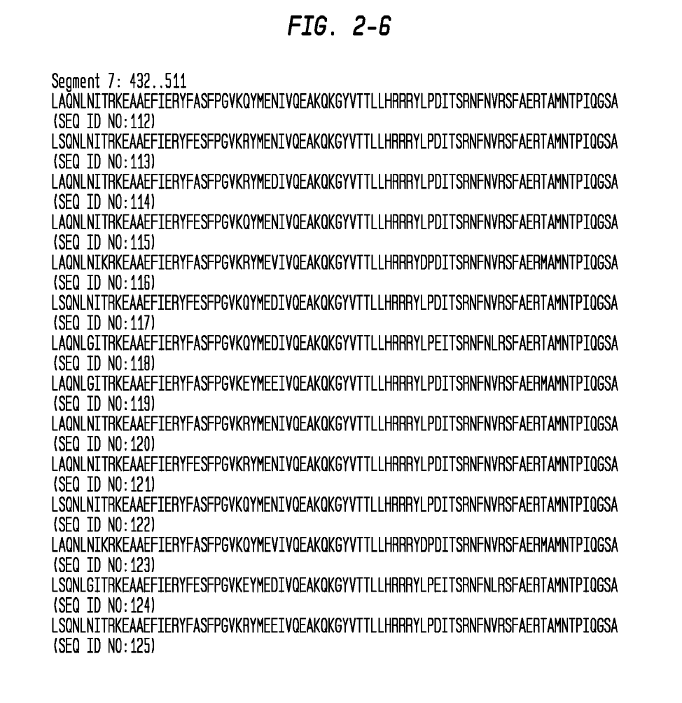

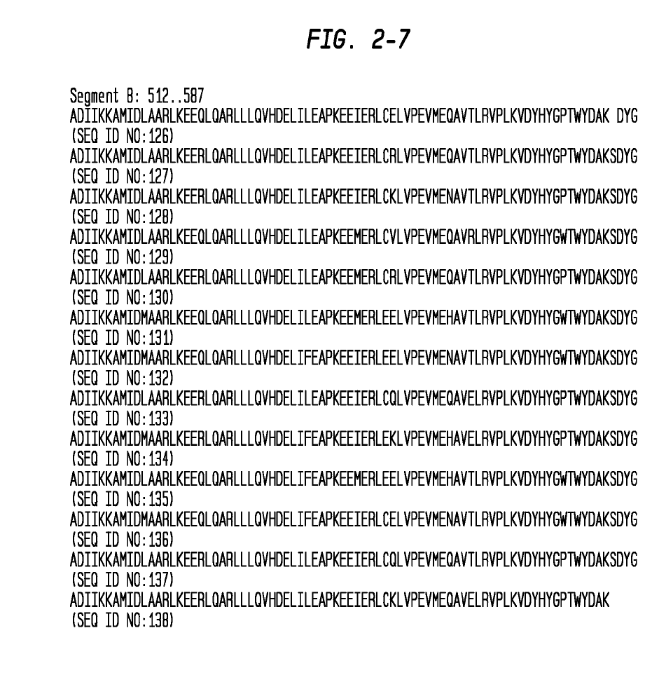

In another embodiment, a synthetic protein is described that contains sequences from single fragments selected from each of 8 segments assembled in order of the 8 numbered segments (see FIG. 2). The synthetic protein may be synthesized either as a single DNA or protein sequence or as a set of polynucleotides or peptides that are ligated together using techniques known in the art (see for example Gibson Assembly™ Master Mix (New England Biolabs, Ipswich, Mass.), U.S. Pat. No. 7,435,572 or U.S. Pat. No. 6,849,428):

a Fragment 1 selected from Segment 1 having an amino acid sequence selected from the group consisting of SEQ ID NOs:24-39;

a Fragment 2 selected from Segment 2 having an amino acid sequence selected from the group consisting of SEQ ID NOs:40-56;

a Fragment 3 selected from Segment 3 having an amino acid sequence selected from the group consisting of SEQ ID NOs:57-72;

a Fragment 4 selected from Segment 4 having an amino acid sequence selected from the group consisting of SEQ ID NOs:73-87;

a Fragment 5 selected from Segment 5 having an amino acid sequence selected from the group consisting of SEQ ID NOs:88-99;

a Fragment 6 selected from Segment 6 having an amino acid sequence selected from the group consisting of SEQ ID NOs:100-111;

a Fragment 7 selected from Segment 7 having an amino acid sequence selected from the group consisting of SEQ ID NOs:112-125;

a Fragment 8 selected from Segment 8 having an amino acid sequence selected from the group consisting of SEQ ID NOs:126-138.

A proviso for creating a synthetic protein is that the synthetic protein has a sequence that differs from any SEQ ID NOs:1-23.

Preferably, a synthetic protein comprising segments 1-8 has at least one, two, three, four, five, six, seven, eight, nine or 10 sequence motifs selected from 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569.

The synthetic proteins described herein and characterized by a non-natural amino acid sequence generally retain DNA binding properties making these synthetic proteins useful for example as DNA detection reagents. The variants may be screened using at least one method described in Examples 1-6, or by other screening methods common used in the art, so as to identify those variants having at least one of the functional properties that are at least typical of a Family A DNA polymerase and/or have one or more improved properties selected from at least one of specific activity; reaction speed; thermostability; storage stability; dUTP tolerance and salt tolerance; increased performance in isothermal amplification; non-interference of pH during sequencing; improved strand displacement; altered processivity; altered ribonucleotide incorporation; altered modified nucleotide incorporation; and altered fidelity when compared to the corresponding parent polymerase. The improved properties of these mutant enzymes have been demonstrated to enhance the performance of sequencing platforms (for example, the Ion Torrent™ sequencer (Life Technologies, Carlsbad, Calif.)). The improved properties of these mutant enzymes enhance their use in isothermal amplification for diagnostic applications.

The DNA polymerase variants and synthetic proteins described herein may be expressed in suitable non-native host cells such as E. coli according to standard methods known in the art. To facilitate expression, the variant DNA polymerase may additionally have a methionine in front of the first amino acid at the N-terminal end. Host cells may be transformed with DNA encoding the variant optionally contained in a suitable expression vector (see New England Biolabs catalog 2019-10 or 2011-12 for expression vectors known in the art for this purpose). Transformation is achieved using methods well known in the art.

The DNA polymerase variants and synthetic proteins characterized herein may further be modified by additions and/or deletions of peptides at their N-terminal and/or C-terminal ends. For example, fusion of a peptide to a synthetic protein may include fusion of one or more of a DNA binding domain (such as Sso7d from archaea), an exonuclease domain (such as amino acids 1-289 of Bst DNA polymerase), a peptide lacking exonuclease activity (for example, a mutated exonuclease domain similar to amino acids 1-289 of Bst DNA polymerase), an affinity binding domain such as a Histidine tag, chitin binding domain, or intein, and a solubility tag such as maltose binding domain (MBP). The addition of a peptide fused to an end of the amino acid sequence of the DNA polymerase may be used to enhance one or more of the functional features described in Examples 1-6. Aptamers may be fused to one end of the mutant DNA polymerase.

The variants may be stored in a storage or reaction buffer that includes a detergent such as a non-ionic detergent, a zwitterionic detergent, an anionic detergent or a cationic detergent. The storage or reaction buffer may further include one or more of: a polynucleotide, for example, an aptamer for facilitating a hot start; polynucleotide primers, dNTPs, target polynucleotides; additional polymerases including additional DNA polymerases; RNA polymerases and/or reverse transcriptases; crowding agents such as polyethylene glycol; and/or other molecules known in the art for enhancing the activity of the DNA polymerase variants.

The DNA polymerase variant and synthetic proteins may be used for DNA synthesis, DNA repair, cloning and sequencing (see for example U.S. Pat. No. 7,700,283 and US Application Publication No. US 2011/0201056) and such as illustrated in the examples and also for temperature dependent amplification methods. Examples of isothermal amplification methods in addition to loop-mediated isothermal amplification (LAMP) used in the present examples include helicase dependent amplification (HDA) (see for example U.S. Pat. No. 7,829,284, U.S. Pat. No. 7,662,594, and U.S. Pat. No. 7,282,328); strand displacement amplification (SDA); nicking enzyme amplification reaction; recombinase polymerase amplification; padlock amplification; rolling circle amplification; and multiple displacement amplification (see for example Gill, et al., Nucleosides, Nucleotides and Nucleic Acids, 27:224-243 (2008)). The variant and synthetic DNA polymerases described herein may also be used in sample preparation for sequencing by synthesis techniques known in the art. The variant and/or synthetic polymerases may also be used in quantitative amplification techniques known in the art that may be performed at a temperature at which the variant or synthetic protein effectively polymerizes nucleotides.

### EXAMPLES

The examples below illustrate assays and properties of Bst DNA polymerase variants described above.

### Example 1

### Assay for Determining the Properties of a Variant DNA Polymerase

(a) Loop-Mediated Isothermal Amplification (LAMP)

The properties of a variant polymerase can be determined using an isothermal amplification procedure such as a LAMP protocol (Nagamine, et al., Mol. Cell. Probes, 16:223-229(2002); Notomi, et al., Nucleic Acids Research, 28:E63 (2000)).

The LAMP reaction used bacteriophage A genomic DNA (New England Biolabs, Ipswich, Mass.) as the template. The LAMP primers used here were:

(SEQ ID NO: 139)
FIP (5′-CAGCCAGCCGCAGCACGTTCGCTCATAGGAGATATGGTAGA
GCCGC-3′),
(SEQ ID NO: 140)
BIP (5′GAGAGAATTTGTACCACCTCCCACCGGGCACATAGCAGTCCT
AGGGACAGT-3′),
(SEQ ID NO: 141)
F3 (5′-GGCTTGGCTCTGCTAACACGTT-3′),
(SEQ ID NO: 142)
B3 (5′-GGACGTTTGTAATGTCCGCTCC-3′),
(SEQ ID NO: 143)
LoopF (5′-CTGCATACGACGTGTCT-3′),
(SEQ ID NO: 144)
LoopB (5′-ACCATCTATGACTGTACGCC-3′).

The LAMP reaction used 0.4 U-0.2 U variant Polymerase/μL, 1.6 μM FIP/BIP, 0.2 μM F3/B3, 0.4 μM LoopF/LoopB, and 5 ng lambda DNA in a buffer containing 1× ThermoPol Detergent-free, 0.1% Tween 20, 6-8 mM MgSO₄ and 1.4 μMdNTP. The reaction was followed by monitoring turbidity in real time using the Loopamp® Realtime Turbidimeter LA-320c (SA Scientific, San Antonio, Tex.) or with a CFX96™ Real-Time fluorimeter (Bio-Rad, Hercules, Calif.). The reaction conditions were varied to determine the optimum range that the variant DNA polymerase could perform LAMP. This was compared with the parent Bst DNA polymerase. The parent Bst DNA polymerase was typically used at 65° C. in these LAMP reaction conditions. However, the temperature was varied to determine the optimum temperature for a particular variant. Different salt conditions and rates of reaction were tested and variants identified which were 10%-50% faster than the parent polymerase and had an increased salt tolerance to as much as 200 mM KCl.

The results are shown in FIG. 4.

(b) DNA Polymerase Activity Assay Using Modified Nucleotides in a Comparison of the Activity of a Fusion Variant Protein with Exonuclease Activity, with Full Length Parent Bst Polymerase.

This assay was used to determine the activity of the variant polymerase having exonuclease activity as a result of an additional 289 amino acid sequence at the N-terminal end that has been described in detail for parent DNA Bst polymerase. The activity was measured by incorporation of a radioactive ³H-dTTP in a DNA substrate using various concentrations of a variant polymerase. A DNA polymerase reaction cocktail (40 μl) was prepared by mixing 30 nM single-stranded M13 mp18, 82 nM primer #1224 (5′-CGCCAGGGTTTTCCCAGTCACGAC-3′) (SEQ ID NO:145), 200 μM dATP, 200 μM dCTP, 200 μM dGTP, and 100 or 200 μM dTTP including 0.6 to 0.8 μCi [3H]-dTTP. The DNA polymerase reaction cocktail was mixed with DNA polymerase (2.2 to 8.7 ng for the parent Bst DNA polymerase (FL), 0.27 to 1 ng for the fusion variant, or 2.5 to 20 ng for the parent BstLF), or water for the no enzyme control, and incubated at 65° C. for 5 minutes. Reactions were halted and precipitated by acid precipitation as follows. A 30 μl aliquot of each reaction was spotted onto 3 mm Whatman discs and immediately submerged into cold 10% Trichloroacetic acid (TCA) in 1 L beaker in an ice bucket. A total counts control was spotted as described but not washed. Filters were washed three times with cold 10% TCA for 10 minutes with vigorous shaking and twice with room temperature 95% isopropanol for 5 minutes. Filters were dried under a heat lamp for 10 minutes and counted using a scintillation counter. The pmoles of dNTPs incorporated were calculated for each sample from the fraction of radioactive counts incorporated, multiplied by the total amount of dNTPs and the volume of the reaction.

A tenfold increase in specific activity of the fusion variant polymerase was found compared with the parent FL Bst polymerase where the fusion variant DNA polymerase was present in the mixture at 506,000 U/mg while the parent Bst DNA polymerase was present at 48,000 U/mg. (1 unit=incorporation of 10 nmol dNTP in 30 minutes at 65° C.).

A 15% increase in activity of the variant polymerase compared with the parent Bst large fragment DNA polymerases was observed in which the variant DNA polymerase was present in the mixture at 370,000 U/mg and the parent BstLF was present at 260,000 U/mg.

### Example 2

### Variant DNA Polymerase Thermostability

The thermostability of the variant DNA polymerase was assessed by incubating the polymerase at differing temperatures followed by performing either one or both of the DNA polymerase assay described in Example 1. The results are shown in FIG. 4C.

### Example 3

### Inhibitor Resistance of the Variant DNA Polymerase

The resistance of a variant DNA polymerase to inhibitors such as blood is determined by adding increasing concentrations of the inhibitor into the DNA polymerase assay and determining the change, if any, in the apparent specific activity of the protein. The DNA polymerase assay was performed as described in Example 1 at 65° C.

Another inhibitor of DNA polymerase is dUTP which is used to prevent carryover contamination in isothermal amplification by replacing dTTP. In this case it is desirable for the polymerase to be insensitive to dUTP inhibition so as to utilize dUTP as a substrate for LAMP. FIG. 4E shows that the mutant polymerase can efficiently utilize dUTP while the wild type Bst polymerase is inhibited by substituting dTTP with dUTP in the amplification reaction.

### Example 4

### Increased Resistance to High Salt Concentration

The resistance of a variant DNA polymerase to increased salt concentration was determined by adding increasing concentrations of salt (for example, KCl or NaCl) to the DNA polymerase assay described in Example 1 and determining the activity of the protein at 65° C. and comparing its activity to parent Bst DNA polymerase (see FIG. 4B).

### Example 5

### Increased Stability in Storage

The stability of a variant DNA polymerase during storage was determined by incubating the enzyme in storage buffer (10 mMTris-HCl pH 7.5, 50 mM KCl, 1 mM Dithiothreitol, 0.1 mM EDTA, 50% Glycerol, 0.1% Triton X-100) at a temperature ranging from 4° C. to 65° C. for a time period ranging from 1 day to 28 weeks, and assaying DNA polymerase activity remaining after storage using the LAMP method described in Example 1. The remaining activity was compared to a sample stored at −20° C. for the same amount of time. The stability of the variant was then compared to the stability of parent Bst DNA polymerase (See FIG. 4D). When this period was extended to 60 weeks, no detectable loss of activity of the mutants was observed even in the absence of glycerol.

### Example 6

### Assay for Determining the Melting Temperature of a Variant Polymerase for Comparison with a Parent DNA Polymerase Using a SYPRO Orange Assay

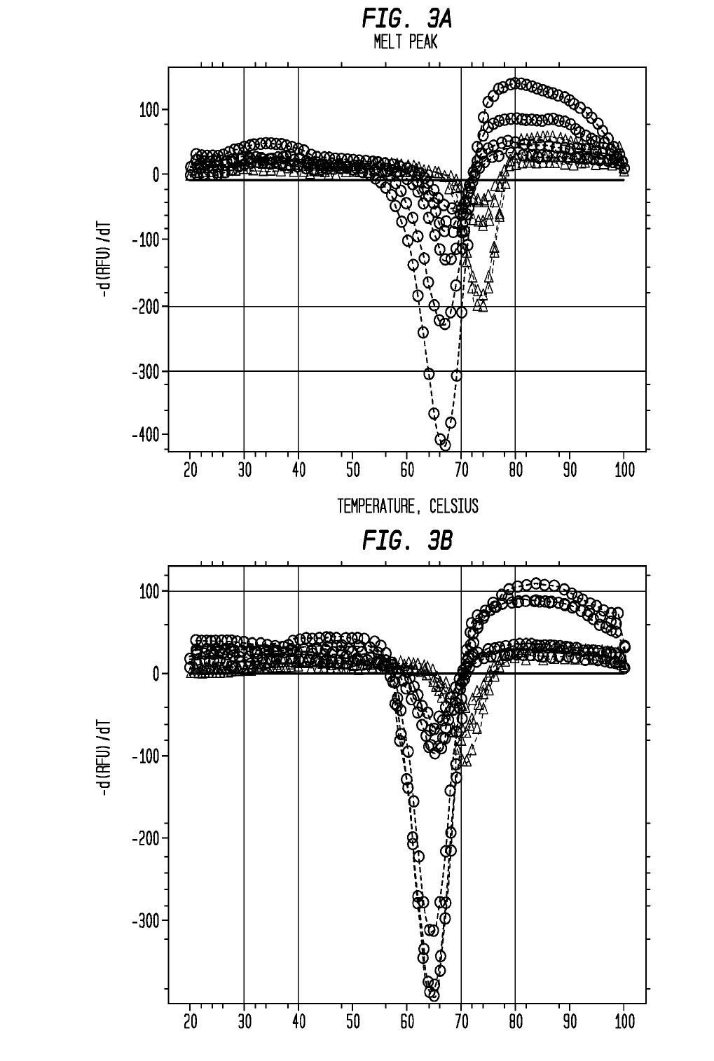

The assay was performed as follows: Each 50 μl reaction contains 1× ThermoPol Buffer, detergent-free (20 mM Tris-HCl pH 8.8, 10 mM (NH₄)₂SO₄, 10 mM KCl, 2 mM MgSO4, 1× SYPRO Orange protein gel stain, and DNA polymerase concentrations ranging from 2.2 to 17.5 μg (parent BstLF mutant) or 0.6 to 4.8 μg (parent Bst FL mutant). The reactions were placed in a CFX96 Real-Time System. The temperature was raised 1° C. per second from 20 to 100° C., and the fluorescence (in the FRET channel) was read at each temperature. Here, the Tm is the inflection point of the sigmodial curve of fluorescence plotted against temperature. The inverted first derivative of the fluorescence emission in FIGS. 3A and 3B is shown in relation to temperature, where the location of the minima corresponded to the value of the Tm (see FIG. 3).

### Example 7

### Whole Genome Amplification Using a Variant Bst DNA Polymerase

The variant DNA polymerase can be tested for suitability in whole genome amplification using the methods termed hyperbranched strand displacement amplification (Lage, et al., Genome Research, 13 (2):294-307 (2003)) or multiple-strand displacement amplification (Aviel-Ronen, et al., BMC Genomics, 7:312 (2006)).

### Example 8

### DNA Sequencing on a Semiconductor Device Using a Variant DNA Polymerase

The variant DNA polymerase can be tested for its suitability in DNA sequencing, for example, as described in Rothberg, et al., Nature, 475(7356):348-352(2011), an integrated semiconductor device enabling non-optical genome sequencing.

### Example 9

### Solid-Phase DNA Amplification Using a Variant Polymerase

Variant DNA polymerase can be tested for its suitability in solid-phase DNA amplification, for example as described in (Adessi, et al., Nucleic Acids Research, 28:E87 (2000), which describes a method for the amplification of target sequences with surface bound oligonucleotides.

### Example 10

### Enhanced Reverse Transcriptase Activity

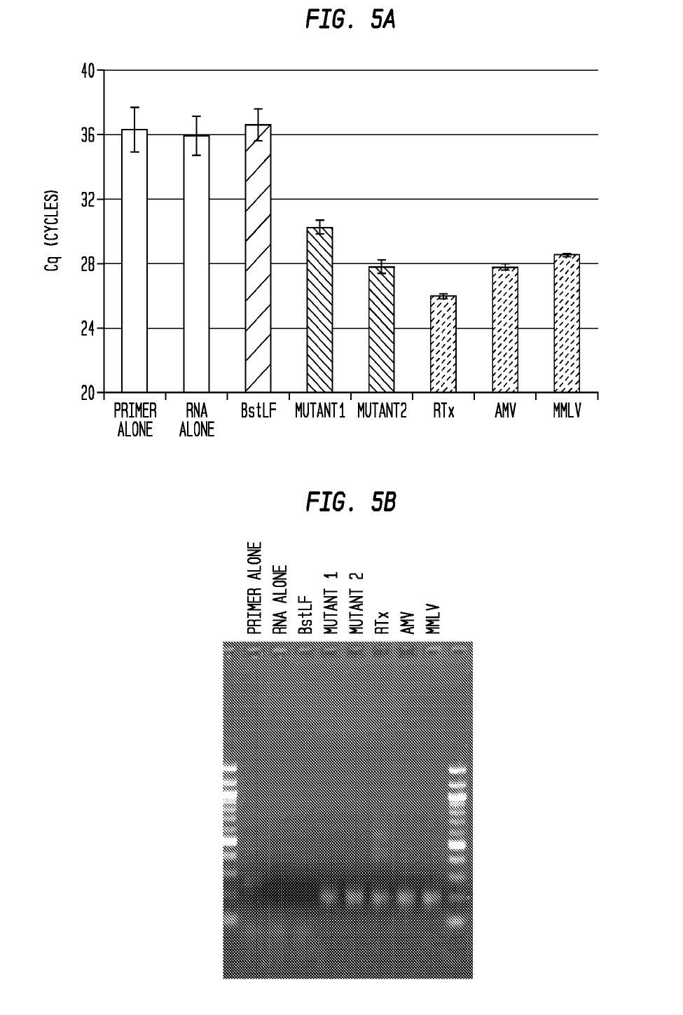

The reverse activity of the mutant Bst DNA polymerase was determined using a two-step RT-qPCR assay (Sambrook, et al., Molecular Cloning—A Laboratory Manual, 3^{rd} ed., Cold Harbor Laboratory Press (2001)). The first step was for cDNA synthesis using the mutant enzymes and various traditional reverse transcriptases. The second measures the amount of synthesized cDNA by qPCR. The RT step was performed using 6 uM Hexamer (Random Primer Mix, New England Biolabs, Ipswich, Mass.) as primers in Isothermal Amplification Buffer (New England Biolabs, Ipswich, Mass.) supplemented with 6 mM Mg and 200 uM dNTP with 0.1 ug Jurkat Total RNA (Life Technologies, Carlsbad, Calif.) and incubated at 65° C. for 20 minutes. 1 ul of the RT product was added to qPCR reaction for GAPDH gene with 200 nM of forward (5′-AGAACGGGAAGCTTGTCATC) (SEQ ID NO:146) and reverse primer (5′-CGAACATGGGGGCATCAG) (SEQ ID NO:147), 200 uM dNTP, 1.25 unit of Taq DNA polymerase in 25 ul of 1× Standard Taq Buffer (New England Biolabs, Ipswich, Mass.) containing 2 uM of dsDNA-binding fluorescent dye SYTO® 9 (Life Technologies, Carlsbad, Calif.). The PCR cycles were: 95° C. for 1 minute, then 50 cycles at 95° C. for 10 seconds, 61° C. for 15 seconds and 68° C. for 30 seconds, and a final step of 68° C. for 5 minutes. The PCR was performed on a CFX96 Real-Time PCR machine and the Cq value was obtained as an indication of the amount of specific cDNA being synthesized (FIG. 5A). Mutant 1 and mutant 2 (4^{th} and 5^{th} bar from left in bar chart) make abundant cDNA as indicated by having Cq values similar to that of traditional RTs (6^{TH}, 7^{th}, and 8^{th} bar from left) in qPCR. Wild type BstLF (3rd bar from the left) is the same as controls (1st and 2^{nd} bar from left) without RT. After completion of the PCR reaction, 10 ul of PCR product was analyzed by electrophoresis in a 1.5% agarose gel (FIG. 5B) to verify the size of the PCR product. The lanes from left to right are primer alone, RNA alone, BstLF, mutant 1, mutant 2, Rtx, AMV and MMLV. Mutant 1, mutant 2 and all RTs (Rtx, AMV and MMLV) lanes gave a band of expected size (207 base pairs) but no specific band with wild type BstLF or controls. These results demonstrate that mutant 1 and mutant 2 has much improved Rtx activity compared to wild type BstLF.

All references cited herein, as well as U.S. provisional application Ser. No. 61/530,273 filed Sep. 1, 2011 and U.S. provisional application Ser. No. 61/605,484 filed Mar. 1, 2012, are herein incorporated by reference.

What is claimed is:

1-50. (canceled)

51. A variant Family A DNA polymerase comprising two or more amino acid sequence motifs selected from 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569, where the number preceding the amino acid in the motif corresponds to the location of that amino acid in the amino acid sequence of FIG. 1.

52. A variant polymerase according to claim 51, comprising at least three or four or five or six or seven or eight or nine or ten or eleven or twelve of said motifs.

53. A variant polymerase according to claim 51, further comprising one or more mutations selected from the group of mutations consisting of (a)-(f) where the mutations in (a)-(f) are:
(a) A1E, G3(K, E or D), K5(L, A or V), E8(M or A), E9D, M10I, A13(D, E, T or V), I14D, V15A, V17(T, E or G), I18V;
(b) E20(M or D) M34(Q or L), E36D, I46F, L48(N or I), M57(L or I), P59(T or A), T61L, D65S, S66(F, E or P);
(c) Q67A, L69(V or K), A73E, M81V, A84R, V88(A or I), R99V, A102D, N113A, D117(T, S or A), A118D, G119(D or E), I121(A or V), V124K, E131H, S135(E or P), V144A, S147(P or A), L148(D or V), Q152(L or P);
(d) T153(A or V), Q170(R or E), M173(L or I), D175E, N178(E, K or R), Q183(L, E or R), L185F, T186(L or I), K187(E or D), Q190(L or M);
(e) A193(I or S), A194(L, S or T), N205(D or K), S216(L or E), R223(V, K or G), A224E, I225(Q or V), V247L, R307H, M316R; and
(f) A330T, D357L, D378N, D380E, I383A, Q387R, L390M; 1400V, E406D, A410S, N411R, A433S, N437G, T439K, A452E, Q459(R or E), N463(V, E or D), L484D, D486E, V494L, T501M, Q530R, I552M, E557(K, Q or R) and T568(E or R).

54. A variant polymerase according to claim 53, comprising at least one mutated amino acid selected from each of groups (a)-(f).

55. A variant polymerase of claim 53, further comprising two, three, four, five, six, seven, eight, nine, ten, eleven, twelve, thirteen, fourteen, fifteen, sixteen, seventeen, eighteen, nineteen, or twenty mutant amino acids at the same position as the corresponding amino acids in (a)-(f) of SEQ ID NO:1.

56. A variant polymerase according to claim 51, wherein a binding domain is fused to one end of the variant polymerase directly or by means of a linker sequence.

57. A variant polymerase according to claim 53, wherein a binding domain is fused to one end of the variant polymerase directly or by means of a linker sequence.

58. A DNA encoding a variant polymerase according to claim 51.

59. A DNA encoding a variant polymerase according to claim 53.

60. A host cell comprising the DNA according to claim 58.

61. A host cell comprising the DNA according to claim 59.

62. A method for reverse transcribing an RNA of interest, comprising combining an RNA with a DNA polymerase variant or preparation thereof according to claim 53, to form a cDNA.

63. A method according to claim 62, further comprising amplifying the cDNA by means of the DNA polymerase variant or preparation thereof.

64. A method for amplifying DNA, comprising combining a target DNA with a DNA polymerase variant or preparation thereof according to claim 53, to produce amplified DNA.

65. A variant protein, comprising: an amino acid sequence with at least 90% but less than 100% sequence identity to any of SEQ ID NOs:1-23, wherein the variant protein further comprises at least one mutated amino acid having a position corresponding to SEQ ID NO:1 selected from the group of mutated amino acids consisting of (a)-(f) where the mutations in (a)-(f) are:
(a) A1E, G3(K, E or D), K5(L, A or V), E8(M or A), E9D, M10I, A13(D, E, T or V), I14D, V15A, V17(T, E or G), I18V;
(b) E20(M or D) M34(Q or L), E36D, I46F, L48(N or I), M57(L or I), P59(T or A), T61L, D65S, S66(F, E or P);
(c) Q67A, L69(V or K), A73E, M81V, A84R, V88(A or I), R99V, A102D, N113A, D117(T, S or A), A118D, G119(D or E), I121(A or V), V124K, E131H, S135(E or P), V144A, S147(P or A), L148(D or V), Q152(L or P);
(d) T153(A or V), Q170(R or E), M173(L or I), D175E, N178(E, K or R), Q183(L, E or R), L185F, T186(L or I), K187(E or D), Q190(L or M);
(e) A193(I or S), A194(L, S or T), N205(D or K), S216(L or E), R223(V, K or G), A224E, I225(Q or V), V247L, R307H, M316R; and
(f) A330T, D357L, D378N, D380E, I383A, Q387R, L390M; 1400V, E406D, A410S, N411R, A433S, N437G, T439K, A452E, Q459(R or E), N463(V, E or D), L484D, D486E, V494L, T501M, Q530R, I552M, E557(K, Q or R) and T568(E or R).

66. The variant protein according to claim 65, further comprising at least one amino acid mutation selected from each of groups (a)-(f).

67. The variant protein according to claim 65, further comprising at least one amino acid motif or at least two amino acid motifs selected from the group consisting of: 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569.

68. A variant protein according to claim 65, further comprising two, three, four, five, six, seven, eight, nine, ten, eleven, twelve, thirteen, fourteen, fifteen, sixteen, seventeen, eighteen, nineteen, or twenty mutations at the same position as the corresponding mutant amino acids in (a)-(f) of SEQ ID NO:1.

69. A non-naturally occurring synthetic protein, comprising: a fragment 1, a fragment 2, a fragment 3, a fragment 4, a fragment 5, a fragment 6, a fragment 7 and a fragment 8 wherein the fragments are covalently linked, and wherein:
the fragment 1 is selected from Segment 1 having an amino acid sequence selected from the group consisting of SEQ ID NOs:24-39;
the fragment 2 is selected from Segment 2 having an amino acid sequence selected from the group consisting of SEQ ID NOs:40-56;
the fragment 3 is selected from Segment 3 having an amino acid sequence selected from the group consisting of SEQ ID NOs:57-72;
the fragment 4 is selected from Segment 4 having an amino acid sequence selected from the group consisting of SEQ ID NOs:73-87;
the fragment 5 selected from Segment 5 having an amino acid sequence selected from the group consisting of SEQ ID NOs: 88-99;
the fragment 6 selected from Segment 6 having an amino acid sequence selected from the group consisting of SEQ ID NOs:100-111;
the fragment 7 selected from Segment 7 having an amino acid sequence selected from the group consisting of SEQ ID NOs:112-125;
the fragment 8 selected from Segment 8 having an amino acid sequence selected from the group consisting of SEQ ID NOs:126-138; and
wherein the covalently linked fragments having an amino acid sequence that does not have 100% identity to SEQ ID NOs:1-23.

70. A synthetic protein according to claim 69, wherein the amino acid sequence of the synthetic protein comprises at least one amino acid sequence motif selected from the group consisting of: 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569.

71. The synthetic protein of claim 69, wherein the amino acid sequence comprises at least two or three or four or five or six or seven or eight or nine or ten or eleven or twelve of the amino acid sequence motifs selected from the group consisting of: 3 . . . EEK . . . 5, 15 . . . ADE . . . 17 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569.

72. A protein comprising at least 90% sequence identity with SEQ ID NO:1 and further comprising one or more mutations (such as, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 mutations) selected from the group consisting of: A1E, G3(K, E or D), K5(L, A or V), E8(M or A), E9D, M10I, A13(D, E, T or V), I14D, V15A, V17(T, E or G), I18V, E20(M or D) M34(Q or L), E36D, I46F, L48(N or I), M57(L or I), P59(T or A), T61L, D65S, S66(F, E or P), Q67A, L69(V or K), A73E, M81V, A84R, V88(A or I), R99V, A102D, N113A, D117(T, S or A), A118D, G119(D or E), I121(A or V), V124K, E131H, 5135(E or P), V144A, 5147(P or A), L148(D or V), Q152(L or P), T153(A or V), Q170(R or E), M173(L or I), D175E, N178(E, K or R), Q183(L, E or R), L185F, T186(L or I), K187(E or D), Q190(L or M), A193(I or S), A194(L, S or T), N205(D or K), 5216(L or E), R223(V, K or G), A224E, I225(Q or V), V247L, R307H, M316R, A330T, D357L, D378N, D380E, I383A, Q387R, L390M, 1400V, E406D, A410S, N411R, A433S, N437G, T439K, A452E, Q459(R or E), N463(V, E or D), L484D, D486E, V494L, T501M, Q530R, I552M, E557(K, Q or R) and T568(E or R); and optionally a sequence motif at a specified position in SEQ ID NO:1 selected from the group consisting of: 3 . . . EEK . . . 5, 15 . . . ADE . . . 17, 65 . . . SPQ . . . 67, 86 . . . RAI . . . 88, 185 . . . LTE . . . 187, 186 . . . TEL . . . 188, 222 . . . LKE . . . 224, 306 . . . VHP . . . 308, 314 . . . HTR . . . 316, 555 . . . LCK . . . 557, 556 . . . CKL . . . 558 and 567 . . . VEL . . . 569.

73. A DNA polymerase according to claim 72, containing at least 12 artificially introduced single amino acid mutations that occur in a three amino acid motif that differs from an amino acid in the corresponding site of a naturally occurring Bst polymerase.

74. A DNA polymerase according to claim 72, having one or more improved properties for isothermal amplification compared with SEQ ID NO:1, selected from the group consisting of:
(a) an increased reaction speed in the range where the increase is at least 10% and as much as 20%; 500% or 1000%;
(b) an increased temperature stability in the range of 50° C. to 100° C., 50° C. to 90° C. or 60° C. to 90° C.;
(c) an increased salt tolerance in the range of 10 mM-1 M, or 20 mM-200 mM or 500 mM monovalent salt;
(d) an increased storage stability at 25° C., retaining at least 50% activity over 45 weeks, over 1 year or over 2 years;
(e) an enhanced dUTP tolerance of the range of an increase of 50% to 100% dUTP; and
(f) an increased reverse transcriptase activity by at least 2 fold.


SEQUENCE LISTING (147 sequences)

SEQ ID NO: 1 - protein, 587 aa, Geobacillus sp. G11MC16
AEGEKPLEEMEFAIVDVITEEMLADKAALVVEVMEENYHDAPIVGIALVNEHGRFFMRPETALADSQFLAWLADETKKKSMFDAKRAVVALKWKGIELRGVAFDLLLAAYLLNPAQDAGDIAAVAKMKQYEAVRSDEAVYGKGVKRSLPDEQTLAEHLVRKAAAIWALEQPFMDDLRNNEQDQLLTKLEQPLAAILAEMEFTGVNVDTKRLEQMGSELAEQLRAIEQRIYELAGQEFNINSPKQLGVILFEKLQLPVLKKTKTGYSTSADVLEKLAPHHEIVENILHYRQLGKLQSTYIEGLLKVVRPDTGKVHTMFNQALTQTGRLSSAEPNLQNIPIRLEEGRKIRQAFVPSEPDWLIFAADYSQIELRVLAHIADDDNLIEAFQRDLDIHTKTAMDIFHVSEEEVTANMRRQAKAVNFGIVYGISDYGLAQNLNITRKEAAEFIERYFASFPGVKQYMENIVQEAKQKGYVTTLLHRRRYLPDITSRNFNVRSFAERTAMNTPIQGSAADIIKKAMIDLAARLKEEQLQARLLLQVHDELILEAPKEEIERLCELVPEVMEQAVTLRVPLKVDYHYGPTWYDAK

SEQ ID NO: 2 - protein, 586 aa, Geobacillus stearothermophilus
AEGRKPLEEMEFAIVDVITEEMLADKAALVVEVMEENYHDAPIVGIALVNEHGRFFMRPETALADSQFLAWLADETKKKSMFDAKRAVVALKWKGIDVRGVAFDLLLAAYLLNPAQDAGDIAAVAKMKQYEAVRSDEAVYGKGVKRSLPDEQTLAEHLVRKAAAIWALEQPFMDDLRNNEQDQLLTKLEQPLAAILAEMEFTGVNVDTKRLEQMGSELAEQLRAIEQRIYEHAGQEFNINSPKQLGVILFEKLQLPVLKKTKTGYSTSADVLEKLAPHHEIVENILHYRQLGKLQSTYIEGLLKVVRPDTGKVHTMFNQTLTQTGRLSSAEPNLQNIPIRLEEGRKIRQAFVPSEPDWLIFAADYSQIELRVLAHIADDDNLIEAFQRDLDIHTKTAMDIFHVSEEEVTANMRRQAKAVNFGIVYGISDYGLAQNLNITRKEAAEFIERYFASFPGVRRYMENIVQEAKQKGYVTTLLHRRRYLPDITSRNFNVRSFAERTAMNTPIQGSAADIIKKAMIDLAARLKEEQLQARLLLQVHDELILEAPKEEIERLCELVPEVMEQAVSSVPLKVDYHYGPTWYDAK

SEQ ID NO: 3 - protein, 587 aa, Geobacillus so, MKK(2005)
AEDETPLMEMEFVIADGITDEMLADKAALVVEVMEENYHDAPIVGIALVNEHGRFFLRAEMALADSQFLAWLADETKKKSMFDAKRAAVALKWKGIELRGVAFDLLLAAYLLNPAQDAGDVAAVAKMKQYEAVRPDEAVYGKGAKRSLPDEPTLAEHLVRKAAAIWALERPFLDELRSNEQDGLLIKLEQPLATILAEMEFTGIKVDTKRLEQMGSELAEQLRAVEQRIYELAGQEFNINSPKQLGIILFEKLQLPVLKKTKTGYSTSADVLEKLAPHHEIVENILHYRQLGKLQSTYIEGLLKVVHPDTGKVHTMFNQALTQTGRLSSAEPNLQNIPIRLEEGRKIRQAFVPSEPDWLIFAADYSQIELRVLAHIADDDNLIEAFRRDLDIHTKTAMDIFHVSEEEVTATMRRQAKAVNFGIVYGISDYGLAQNLNITRKEAAEFIERYFASFPGVKRYMETIVQEAKQKGYVTTLLHRRRYLPDITSRNFNVRSFAERTAMNTPIQGSAADIIKKAMIDLAARLKEERLQARLLLQVHDELILEAPKEEMERLCQLVPEVMEQAVALRVPLKVDYHYGPTWYDAK

SEQ ID NO: 4 - protein, 587 aa, Geobacillus sp. LH8
AEDETPLMEMEFVIADGITDEMLADKAALVVEVMEENYHDAPIVGIALVNEHGRFFLRAEMALADSQFLAWLADETKKKSMFDAKRAAVALKWKGIELRGVAFDLLLAAYLLNPAQDAGDVAAVAKMKQYEAVRPDEAVYGKGAKRSLPDEPTLAEHLVRKAAAIWALERPSLDELRSNEQDGLLIKLEQPLATILAEMEFTGIKVDTKRLEQMGSELAEQLRAVEQRIYELAGQEFNINSPKQLGIILFEKLQLPVLKKTKTGYSTSADVLEKLAPHHEIVENILHYRQLGKLQSTYIEGLLKVVHPDTGKVHTMFNQALTQTGRLSSAEPNLQNIPIRLEEGRKIRQAFVPSEPDWLIFAADYSQIELRVLAHIADDDNLIEAFRRDLDIHTKTAMDIFHVSEEEVTATMRRQAKAVNFGVVYGISDYGLAQNLNITRKEAAEFIERYFASFPGVKRYMETIVQEAKQKGYVTTLLHRRRYLPDITSRNFNVRSFAERTAMNTPIQGSAADIIKKAMIDLAARLKEERLQARLLLQVHDELILEAPKEEMERLCQLVPEVMEQAVALRVPLKVDYHYGPTWYDAK

SEQ ID NO: 5 - protein, 587 aa, Geobacillus kaue
AEDETPLMEMEFVAADGITDEMLADKAALVVEVMEENYHDAPIVGIALVNEHGRFFLRAEMALADPQFVAWLADETKKKSMFDAKRASVALKWKGIELRGVAFDLLLAAYLLNPAQDAGDVAAVAKMKQYEAVRPDEAVYGKGAKRSLPDEPTLAEHLVRKAAAIWALERPFLDELRSNEQDELLIKLEQPLATILAEMEFTGVKVDTKRLEQMGSELAEQLGAIEQRIYELAGQEFNINSPKQLGIILFEKLQLPVLKKTKTGYSTSADVLEKLAPHHEIVENILHYRQLGKLQSTYIEGLLKVVHHDTGKVHTMFNQALTQTGRLSSAEPNLQNIPIRLEEGRKIRQAFVPSEPGWLIFAADYSQIELRVLAHIADDDNLIEAFRRDLDIHAKTAMDIFHVSEEEVTATMRRQAKAVNFGIVYGISDYGLAQNLNITRKEAAEFIERYFASFPGVKRYMETIVQEAKQKGYVTTLLHRRRYLPDITSRNFNVRSFAERTAMNTPIQGSAADIIKKAMIDLAARLKEERLQARLLLQVHDELILEAPKEEMERLCQLVPEVMEQAVELRVPLKVDYHYGPTWYDAK

SEQ ID NO: 6 - protein, 587 aa, Geobacillus anatolicus
AEDETPLMEMEFVAADGITDEMLADKAALVVEVMEENYHDAPIVGIALVNEHGRFFLRAEMALADPQFVAWLADETKKKSMFDAKRASVALKWKGIELRGVAFDLLLAAYLLNPAQDAGDVAAVAKMKQYEAVRPDEAVYGKGAKRSLPDEPTLAEHLVRKAAAIWALERPFLDELRSNEQDELLIKLEQPLATILAEMEFTGVKVDTKRLEQMGSELAEQLGAVEQRIYELAGQEFNINSPKQLGIILFEKLQLPVLKKTKTGYSTSADVLEKLAPHHEIVENILHYRQLGKLQSTYIEGLLKVVHHDTGKVHTMFNQALTQTGRLSSAEPNLQNIPIRLEEGRKIRQAFVPSEPGWLIFAADYSQIELRVLAHIADDDNLIEAFRRDLDIHTKTAMDIFHVSEEEVTATMRRQAKAVNFGIVYGISDYGLAQNLNITRKEAAEFIERYFASFPGVKRYMETIVQEAKQKGYVTTLLHRRRYFPDITSRNFNVRSFAERTAMNTPIQGSAADIIKKAMIDLAARLKEERLQARLLLQVHDELILEAPKEEMERLCQLVPEVMEQAVELRVPLKVDYHYGPTWYDPK

SEQ ID NO: 7 - protein, 587 aa, Geobacillus bogazici
AEDETPLMEMEFVAADGITDEMLADKAALVVEVMEENYHDAPIVGIALVNEHGRFFLRAEMALADPQFVAWLADETKKKSMFDAKRASVALKWKGIELRGVAFDLLLAAYLLNPAQDAGDVAAVAKMKQYEAVRPDEAVYGKGAKRSLPDEPTLAEHLVRKAAAIWALERPFLDELRSNEQDELLIKLEQPLATILAEMEFTGVKVDTKRLEQMGSELAEQLGAVEQRIYELAGQEFNINSPKQLGIILFEKLQLPVLKKTKTGYSTSADVLEKLAPHHEIVENILHYRQLGKLQSTYIEGLLKVVHHDTGKVHTMFNQALTQTGRLSSAEPNLQNIPIRLEEGRKIRQAFVPSEPGWLIFAADYSQIELRVLAHIADDDNLIEAFRRDLDIHTKTAMDIFHVSEEEVTATMRRQAKAVNFGIVYGISDYGLAQNLNITRKEAAEFIERYFASFPGVKRYMENIVQEAKQKGYVTTLLHRRRYLPDITSRNFNVRSFAERMAMNTPIQGSAADIIKKAMIDLAARLKEERLQARLLLQVHDELILEAPKEEMERLCRLVPEVMEQAVELRVPLKVDYHYGPTWYDPK

SEQ ID NO: 8 - protein, 587 aa, Geobacillus kaue
AEDETPLMEMEFVAADGITDEMLADKAALVVEVMEENYHDAPIVGIALVNEHGRFSLRAEMALADPQFVAWLADETKKKSMFDAKRASVALKWKGIELRGVAFDLLLAAYLLNPAQDAGDVAAVAKMKQYEAVRPDEAVYGKGAKRSLPDEPTLAEHLVRKAAAIWALERPFLDELRSNEQDELLIKLEQPLATILAEMEFTGVKVDTKRLEQMGSELAEQLGAIEQRIYELAGQEFNINSPKQLGIILFEKLQLPVLKKTKTGYSTSADVLEKLAPHHEIVENILHYRQLGKLQSTYIEGLLKVVHHDTGKVHTMFNQALTQTGRLSSAEPNLQNIPIRLEEGRKIRQAFVPSEPGWLIFAADYSQIELRVLAHIADDDNLIEAFRRDLDIHTKTAMDIFHVSEEEVTATMRRQAKAVNFGIVYGISDYGLAQNLNITRKEAAEFIERYFASFPGVKRYMETIVQEAKQKGYVTTLLHRRRYFPDITSRNFNVRSFAERTAMNTPIQGSAADIIKKAMIDLAARLKEERLQARLLLQVHDELILEAPKEEMERLCQLVPEVMEQAVELRVPLKVDYHYGPTWYDPK

SEQ ID NO: 9 - protein, 587 aa, Anoxybacillus sp. NB
AEDETPLMEMEFVAADGITDEMLADKAALVVEVMEENYHDAPIVGIALVNEHGRFSLRAEMALADPQFVAWLADETKKKSMFDAKRASVALKWKGIELRGVAFDLLLAAYLLNPAQDAGDVAAVAKMKQYEAVRPDEAVYGKGAKRSLPDEPTLAEHLVRKAAAIWALERPFLDELRSNEQDELLIKLEQPLATILAEMEFTGVKVDTKRLEQMGSELAEQLGAVEQRIYELAGQEFNINSPKQLGIILFEKLQLPVLKKTKTGYSTSADVLEKLAPHHEIVENILHYRQLGKLQSTYIEGLLKVVHHDTGKVHTMFNQALTQTGRLSSAEPNLQNIPIRLEEGRKIRQAFVPSEPGWLIFAADYSQIELRVLAHIADDDNLIEAFRRDLDIHTKTAMDIFHVSEEEVTATMRRQAKAVNFGIVYGISDYGLAQNLNITRKEAAEFIERYFASFPGVKRYMETIVQEAKQKGYVTTLLHRRRYFPDITSRNFNVRSFAERTAMNTPIQGSAADIIKKAMIDLAARLKEERLQARLLLQVHDELILEAPKEEMERLCQLVPEVMEQAVELRVPLKVDYHYGPTWYDPK

SEQ ID NO: 10 - protein, 587 aa, Geobacillus stearothermophilus
DEGEKPLAGMDFAIADSVTDEMLADKAALVVEVVGDNYHHAPIVGIALANERGRFFLRPETALADPKFLAWLGDETKKKTMFDSKRAAVALKWKGIELRGVVFDLLLAAYLLDPAQAAGDVAAVAKMHQYEAVRSDEAVYGKGAKRTVPDEPTLAEHLVRKAAAIWALEEPLMDELRRNEQDRLLTELEQPLAGILANMEFTGVKVDTKRLEQMGAELTEQLQAVERRIYELAGQEFNINSPKQLGTVLFDKLQLPVLKKTKTGYSTSADVLEKLAPHHEIVEHILHYRQLGKLQSTYIEGLLKVVHPVTGKVHTMFNQALTQTGRLSSVEPNLQNIPIRLEEGRKIRQAFVPSEPDWLIFAADYSQIELRVLAHIAEDDNLIEAFRRGLDIHTKTAMDIFHVSEEDVTANMRRQAKAVNFGIVYGISDYGLAQNLNITRKEAAEFIERYFASFPGVKQYMDNIVQEAKQKGYVTTLLHRRRYLPDITSRNFNVRSFAERTAMNTPIQGSAADIIKKAMIDLSVRLREERLQARLLLQVHDELILEAPKEEIERLCRLVPEVMEQAVTLRVPLKVDYHYGPTWYDAK

SEQ ID NO: 11 - protein, 587 aa, Geobacillus stearothermophilus
DEGEKPLAGMDFAIADSVTDEMLADKAALVVEVVGDNYHHAPIVGIALANERGRFFLRPETALADPKFLAWLGDETKKKTMFDSKRAAVALKWKGIELRGVVFDLLLAAYLLDPAQAAGDVAAVAKMHQYEAVRSDEAVYGKGAKRTVPDEPTLAEHLARKAAAIWALEEPLMDELRRNEQDRLLTELEQPLAGILANMEFTGVKVDTKRLEQMGAELTEQLQAVERRIYELAGQEFNINSPKQLGTVLFDKLQLPVLKKTKTGYSTSADVLEKLAPHHEIVEHILHYRQLGKLQSTYIEGLLKVVHPVTGKVHTMFNQALTQTGRLSSVEPNLQNIPIRLEEGRKIRQAFVPSEPDWLIFAADYSQIELRVLAHIAEDDNLIEAFRRGLDIHTKTAMDIFHVSEEDVTANMRRQAKAVNFGIVYGISDYGLAQNLNITRKEAAEFIERYFASFPGVKQYMDNIVQEAKQKGYVTTLLHRRRYLPDITSRNFNVRSFAERTAMNTPIQGSAADIIKKAMIDLSVRLREERLQARLLLQVHDELILEAPKEEIERLCRLVPEVMEQAVALRVPLKVDYHYGPTWYDAK

SEQ ID NO: 12 - protein, 587 aa, Bacillus sp. G(2006)
SEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRSDEAVYGKGAKRAVPDEPVLAEHLVRKAAAIWALERPFLDELRRNEQDRLLVELEQPLSSILAEMEFAGVKVDTKRLEQMGEELAEQLRTVEQRIYELAGQEFNINSPKQLGVILFEKLQLPILKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLQVHDELILEAPKEEMERLCRLVPEVMEQAVTLRVPLKVDYHYGPTWYDAK

SEQ ID NO: 13 - protein, 587 aa, Bacillus caldolyticus
SEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRSDEAVYGKGAKRAVPDEPVLAEHLVRKAAAIWALERPFLDELRRNEQDRLLVELEQPLSSILAEMEFAGVKVDTKRLEQMGEELAEQLRTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLQVHDELILEAPKEEMERLCRLVPEVMEQAVTLRVPLKVDYHYGSTWYDAK

SEQ ID NO: 14 - protein, 587 aa, Geobacillus sp. C56-T3
SEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWALERPFLDELRRNEQDRLLVELEQPLSSILAEMEFAGVKVDTKRLEQMGEELAEQLRTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLQVHDELILEAPKEEMERLCRLVPEVMEQAVTLRVPLKVDYHYGSTWYDAK

SEQ ID NO: 15 - protein, 587 aa, Geobacillus sp. Y412MC61
SEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWALERPFLDELRRNEQDRLLVELEQPLSSILAEMEFAGVKVDTKRLEQMGEELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLQVHDELILEAPKEEMERLCRLVPEVMEQAVTLRVPLKVDYHYGSTWYDAK

SEQ ID NO: 16 - protein, 587 aa, Geobacillus kaustophilus HTA426
SEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLQVHDELILEAPKEEMERLCRLVPEVMEQAVTLRVPLKVDYHYGSTWYDAK

SEQ ID NO: 17 - protein, 588 aa, Bacillus caldotenax
SEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWALERPFLDELRRNEQDRLLVELEQPLSSILAEMEFAGVKVDTKRLEQMGEELAEQLRTVEQRIYELAGQEFNINSPKQLGVILFEKLQLPVLKKSKTGYSTSADVLEKLAPYHEIVENILQHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLQVHDELILEAPKEEMERLCRLVPEVMEQAVTLRVPLKVDYHYGSTWYDAK

SEQ ID NO: 18 - protein, 589 aa, Geobacillus stearothermophilus
DEGEKPLAGMDFAIADSVTDEMLADKAALVVEVVGDNYHHAPIVGIALANERGRFFLRPETAVADPKFLAWLGDETKKKTMFDSKRAAVALNGKGIELAGVGVVFDLLLAAYLLDPAQAAGDVAAVAKMHQYEAVRSDEAVYGKGAKRTVPDEPTLAEQLVRKAAAIWALEEPLMDELRRNEQDRLLTELEHALAGILANMEFTGVKVDTKRLEQMGAELTEQLQAVERRIYELAGQEFNINSPKQLGTVLFDKLQLPVLKKTKTGYSTSADVLEKLAPHHEIVEHILHYRQLGKLQSTYIEGLLKVVHPVTGKVHTMFNQALTQTGRLSSVEPNLQNIPIRLEEGRKIRQAFVPSEPDWLIFAADYSQIELRVLAHIAEDDNLIEAFRRWLDIHTKTAMDIFHVSEEDVTANMRRQAKAVNFGIVYGISDYGLAQNLNITRKEAAEFIERYFASFPGVKQYMDNIVQEAKQKGYVTTLLHRRRYLPDITSRNFNVRTFAERTAMNTPIQGSAADIIKKAMIDLSVSVREERLQARLLLQGHDELILEAPKEEIGRLCRLVPEVMEQAVTLRVPLKVDYHYGPTWYDAK

SEQ ID NO: 19 - protein, 587 aa, Geobacillus sp. WCH70
EETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 20 - protein, 587 aa, Geobacillus caldoxylosilyticus
KKEEAVLPNIDYTIVEEVSESVLADRSALVVEVLESNYHKAPILGFALANEHGHFFIRTDTALSSPLFTAWLEDETKKKSVFDGKRAIVALKWKGIELRGIEFDLLIASYLLNPSQSTEDVASVAKTKQYMAVQSDEAVYGKGAKQTVPEEKALAEHLVRKAAAIRALEKEFIHDLQENEQYALFTDLELPLSSILAEMEFTGVKVDVERLKEMGEELTEQLRAVEQEIYELAGQEFNINSPKQLGVILFEKLQLPVLKKTKTGYSTSADVLEKLAPQHEIVEKILHYRQLGKLQSTYIEGLLKVVHCDTHKVHTIFNQALTQTGRLSSTEPNLQNIPIRLEEGRKIRQAFVPSKPDWVIFSADYSQIELRVLAHIANDENLIDAFRHDLDIHTKTAMDIFHVTADEVTPNMRRQAKAVNFGIVYGISDYGLSQNLNIPRKEAAEFIRRYFEIFPGVKQYMENIVQEAKQKGYVTTLLHRRRYLPDITSRNFNLRSFAERTAMNTPIQGSAADIIKKAMIDLADRLKQEKLQARMLLQVHDELILEAPKEEVKRLQQIVPEVMENAIQLKVPLLIDFRFGPTWDDAK

SEQ ID NO: 21 - protein, 587 aa, Geobacillus sp. Y4.1MC1
EKTAVALPKIRYEIVDEVTEAILSDEAALVVEVLESNYHKAPILGFAIANEHGNFFIRTDTALSSSLFTTWLEDESKKKSVFDGKRAIVSLKWQGVHLRGIQFDLLIASYLLNPSQSTEDVASIAKTKQYAGVQSDEAVYGKGAKQKVPDEQVLAEHLVRKAAAIRALEQGFIHDLQENEQYSLFTDLELPLSTILAEMEFAGVKVDVERLKEMGEELAEQLKEVEQEIYRLAGQEFNINSPKQLGVILFEKLQLPVLKKTKTGYSTSAEVLEKLAPQHEIVEKILHYRQLGKLQSTYIEGLLKVVHRDTNKVHTIFNQALTQTGRLSSTEPNLQNIPIRLEEGRKIRQAFIPSEPDWVIFSADYSQIELRVLAHIANDENLIDAFRHGLDIHTKTAMDIFHVSKEEVTPNMRRQAKAVNFGIVYGISDYGLSQNLNITRKEAAEFIKRYFEIFPGVKQYMKDIVQEAKQKGYVTTLLHRRRYLPDITSRNFNLRSFAERTAMNTPIQGSAADIIKKAMIDLSKRLQKENMKARMLLQVHDELILEAPKDEIERLQQIVPEVMENAVQLRVPLKVDYHFGPTWYDAK

SEQ ID NO: 22 - protein, 587 aa, Geobacillus thermoglucosidasius C56-YS93
EKTAVALPEIGYKIVDEVTEAILSDEAALVVEVLESNYHKAPILGFAIANEHGNFFIRTDTALSSSLFTTWLEDESKKKSVFDGKRAIVSLKWQGVHLRGIQFDLLIASYLLNPSQSTEDVASIAKTKQYVGVQSDEAVYGKGAKQKVPDEQVLAEHLVRKAAAIRALEQGFIHDLQENEQYSLFTDLELPLSTILAEMEFAGVKVDVERLKEMGEELAEQLKEVEQEIYRLAGQEFNINSPKQLGVILFEKLQLPVLKKTKTGYSTSAEVLEKLAPQHEIVEKILHYRQLGKLQSTYIEGLLKVVHRDTNKVHTIFNQALTQTGRLSSTEPNLQNIPIRLEEGRKIRQAFIPSEPDWVIFSADYSQIELRVLAHIANDENLIDAFRHGLDIHTKTAMDIFHVSKEEVTPNMRRQAKAVNFGIVYGISDYGLSQNLNITRKEAAEFIKRYFEIFPGVKQYMKDIVQEAKQKGYVTTLLHRRRYLPDITSRNFNLRSFAERTAMNTPIQGSAADIIKKAMIDLSKRLQKENMKARMLLQVHDELILEAPKDEIERLQQIVPEVMENAVQLRVPLKVDYHFGPTWYDAK

SEQ ID NO: 23 - protein, 587 aa, Anoxybacillus flavithermus WK1
SQEQLSLTDISFVTVQTIDEHMLTKEGALVVEVLDENYHQAPIVGFALVNERGHFFIPTDIALASSRFKRWLEDEQCKKSVFDAKRAIVALKWNGIELKGVDFDLLLAAYLLNPTDANGDVAAVAKTKQYTDVQSDEEVYGKGAKQAIPPTNVLAEHLVRKAKAIASLKETYIQELKRNEQFELLVHLELPLTFILAQMEFYGVKVDVDRLEQMGKEFTAQLEQIEQRIYELAGTTFNINSPKQLGTILFEKLQLPIVKKTKTGYSTSADVLEKLAPYHEIIEQILHYRQLGKLQSTYVEGLMKVVRKDTGKVHTIFQQALTQTGRLSSTEPNLQNIPIRIEEGRKIRQAFVPSSDDWVIFAADYSQIELRVLAHIANDENLIAAFHHDLDIHTKTAMDIFHVKEDEVTAHMRRQAKAVNFGIVYGISDYGLSQNLGITRKEAAEFIERYFRSYPGVKRYMEEVVQDAKQKGYVTTLLHRRRYLPDITSGNFNVRSFAERTAMNTPIQGSAADIIKKAMIDLANRLHEERLQTRLLLQVHDELILEAPKEEIELLKKIVPDVMENAVSLRVPLKVDYHFGPTWYDAK

SEQ ID NO: 24 - protein, 31 aa, Artificial Sequence
  Synthetic construct
AEGEKPLEEMEFAIVDVITEEMLADKAALVV

SEQ ID NO: 25 - protein, 31 aa, Artificial Sequence
  Synthetic construct
AEEEKPLEDIEFEIADEVTEEMLADEAALVV

SEQ ID NO: 26 - protein, 31 aa, Artificial Sequence
  Synthetic construct
AEEEVPLEEMEFVIADEITEEMLADKAALVV

SEQ ID NO: 27 - protein, 31 aa, Artificial Sequence
  Synthetic construct
AEEEVPLEEMEFTIADEITEEMLADKAALVV

SEQ ID NO: 28 - protein, 31 aa, Artificial Sequence
  Synthetic construct
AEEEKPLEEMEFAIADEVTEEMLADKAALVV

SEQ ID NO: 29 - protein, 31 aa, Artificial Sequence
  Synthetic construct
AEDEKPLEEIEFAIADEITEEMLADKAALVV

SEQ ID NO: 30 - protein, 31 aa, Artificial Sequence
  Synthetic construct
AEEEKPLAEMEFTIADEVTEEMLADKAALVV

SEQ ID NO: 31 - protein, 31 aa, Artificial Sequence
  Synthetic construct
AEEEVPLAEMEFVIADEITEEMLADKAALVV

SEQ ID NO: 32 - protein, 31 aa, Artificial Sequence
  Synthetic construct
AEGEKPLAEMEFAIVDEITEEMLADKAALVV

SEQ ID NO: 33 - protein, 31 aa, Artificial Sequence
  Synthetic construct
AEEEAPLEDIEFDIADEVTEEMLADKAALVV

SEQ ID NO: 34 - protein, 31 aa, Artificial Sequence
  Synthetic construct
AEEEKPLAEMEFAIADEVTEEMLADKAALVV

SEQ ID NO: 35 - protein, 31 aa, Artificial Sequence
  Synthetic construct
EEEEVPLEEIEFAIADEVTEEMLADKAALVV

SEQ ID NO: 36 - protein, 31 aa, Artificial Sequence
  Synthetic construct
AEDEKPLAEMEFVIADGITDEMLADKAALVV

SEQ ID NO: 37 - protein, 31 aa, Artificial Sequence
  Synthetic construct
AEKELPLMEMEFADADTITMEMLADKAALVV

SEQ ID NO: 38 - protein, 31 aa, Artificial Sequence
  Synthetic construct
AEEEKPLAEMEFVIADGITEEMLADKAALVV

SEQ ID NO: 39 - protein, 31 aa, Artificial Sequence
  Synthetic construct
AEEEKPLAEMEFAIADEITEEMLADKAALVV

SEQ ID NO: 40 - protein, 80 aa, Artificial Sequence
  Synthetic construct
EVMEENYHDAPIVGIALVNEHGRFFMRPETALADSQFLAWLADETKKKSMFDAKRAVVALKWKGIELRGVAFDLLLAAYL

SEQ ID NO: 41 - protein, 80 aa, Artificial Sequence
  Synthetic construct
EVQEENYHDAPIVGIAIVNEHGRFFLRAETALADFQFVAWLEDETKKKSMFDAKRAVVALKWKGIELVGVDFDLLLAAYL

SEQ ID NO: 42 - protein, 80 aa, Artificial Sequence
  Synthetic construct
EVLEENYHDAPIVGIALVNEHGRFFLRPETALADPQFVAWLEDETKKKSMFDAKRAAVALKWKGIELRGVAFDLLLAAYL

SEQ ID NO: 43 - protein, 80 aa, Artificial Sequence
  Synthetic construct
EVMEENYHDAPIVGIALVNEHGRFFLRAETALADPQFVAWLADETKKKSMFDAKRAAVALKWKGIELRGVAFDLLLAAYL

SEQ ID NO: 44 - protein, 80 aa, Artificial Sequence
  Synthetic construct
EVMEENYHDAPIVGIALVNEHGRFFLRPETALADPQFLAWLADETKKKSMFDAKRAAVALKWKGIELRGVAFDLLLAAYL

SEQ ID NO: 45 - protein, 80 aa, Artificial Sequence
  Synthetic construct
EVLEENYHDAPIVGFAIVNEHGRFFLRTELALADFQFVAWLEDETKKKSMFDRKRAVVALKWKGIELVGVDFDLLLAAYL

SEQ ID NO: 46 - protein, 80 aa, Artificial Sequence
  Synthetic construct
EVQEDNYHDAPIVGFAIVNEHGRFFIRTETALASEAFKAWLADETKKKSVFDAKRAIVALKWKGIELRGVDFDLLLAAYL

SEQ ID NO: 47 - protein, 80 aa, Artificial Sequence
  Synthetic construct
EVMEENYHDAPIVGIALVNEHGRFFLRPETALADPQFLAWLADETKKKSMFDAKRAIVALKWKGIELRGVAFDLLLAAYL

SEQ ID NO: 48 - protein, 80 aa, Artificial Sequence
  Synthetic construct
EVMEENYHDAPIVGIANVNEHGRFFLRTELALADFQFVAWLEDETKKKSMFDRKRAAVALKWKGIELVGVDFDLLLAAYL

SEQ ID NO: 49 - protein, 80 aa, Artificial Sequence
  Synthetic construct
EVLEENYHDAPIVGFALVNEHGRFFIRTETALASSQFKAWLEDETKKKSVFDAKRAIVALKWKGIELRGVDFDLLLAAYL

SEQ ID NO: 50 - protein, 80 aa, Artificial Sequence
  Synthetic construct
EVLEENYHDAPIVGFALVNEHGRFFLRPETALADEQFVAWLADETKKKSVFDAKRAVVALKWKGIELRGVDFDLLLAAYL

SEQ ID NO: 51 - protein, 80 aa, Artificial Sequence
  Synthetic construct
EVMEENYHDAPIVGIALVNEHGRFFLRPETALADPQFVAWLADETKKKSMFDAKRAAVALKWKGIELRGVAFDLLLAAYL

SEQ ID NO: 52 - protein, 80 aa, Artificial Sequence
  Synthetic construct
EVMEENYHDAPIVGIALVNEHGRFFLRPETALASPQFKAWLADETKKKSMFDAKRAIVALKWKGIELRGVAFDLLLAAYL

SEQ ID NO: 53 - protein, 80 aa, Artificial Sequence
  Synthetic construct
EVLEENYHDAPIVGFAIVNEHGRFFIRPETALASSQFKAWLEDETKKKSMFDAKRAAVALKWKGIELRGVAFDLLLAAYL

SEQ ID NO: 54 - protein, 80 aa, Artificial Sequence
  Synthetic construct
EVMEENYHDAPIVGIALVNEHGRFFLRTETALADPQFKAWLADETKKKSMFDAKRAIVALKWKGIELRGVDFDLLLAAYL

SEQ ID NO: 55 - protein, 80 aa, Artificial Sequence
  Synthetic construct
EVQEDNYHDAPIVGFANVNEHGRFFLRTETALASEAFAAWLEDETKKKSVFDAKRAVVALKWKGIELVGVAFDLLLAAYL

SEQ ID NO: 56 - protein, 80 aa, Artificial Sequence
  Synthetic construct
EVLEENYHDAPIVGIALVNEHGRFFLRPETALADSQFLAWLEDETKKKSMFDAKRAAVALKWKGIELRGVAFDLLLAAYL

SEQ ID NO: 57 - protein, 80 aa, Artificial Sequence
  Synthetic construct
LNPAQDAGDIAAVAKMKQYEAVRSDEAVYGKGVKRSLPDEQTLAEHLVRKAAAIWALEQPFMDDLRNNEQDQLLTKLEQP

SEQ ID NO: 58 - protein, 80 aa, Artificial Sequence
  Synthetic construct
LNPAQDAGDVAAVAKMKQYEAVRSDEAVYGKGAKRSLPDEPVLAEHLVRKAAAIWALERPFLDELRENEQDELLTDLEQP

SEQ ID NO: 59 - protein, 80 aa, Artificial Sequence
  Synthetic construct
LNPAQAAGDVAAVAKMKQYEAVRSDEAVYGKGAKRAVPDEPVLAEHLVRKAAAIWALEEPFIDELRRNEQDRLLTDLEQP

SEQ ID NO: 60 - protein, 80 aa, Artificial Sequence
  Synthetic construct
LNPAQDAGDVAAVAKMKQYEAVRSDEAVYGKGAKRAVPDEPTLAEHLVRKAAAIWALERPFLDELRRNEQDELLIKLEQP

SEQ ID NO: 61 - protein, 80 aa, Artificial Sequence
  Synthetic construct
LAPAQDDGDAAAKAKMKQYEAVREDEAVYGKGAKRPDPDELALAEHLVRKAAAIWALERPFLDELRENEQDLLLLELEQP

SEQ ID NO: 62 - protein, 80 aa, Artificial Sequence
  Synthetic construct
LNPAQDAGDVAAVAKMKQYEAVRSDEAVYGKGAKRSLPDEPTLAEHLVRKAAAIWALERPFLDELRENEQDELLIKLEQP

SEQ ID NO: 63 - protein, 80 aa, Artificial Sequence
  Synthetic construct
LNPAQTADDVAAVAKMKQYHAVRSDEAVYGKGAKRAVPDEPVLAEHLVRKAAAIWALEEPFLDELRKNEQDELFTELELP

SEQ ID NO: 64 - protein, 80 aa, Artificial Sequence
  Synthetic construct
LNPAQDAGDVAAVAKMKQYEAVRSDEAVYGKGAKRSLPDEPTLAEHLVRKAAAIWALEQPFIDELRRNEQDELLTKLEQP

SEQ ID NO: 65 - protein, 80 aa, Artificial Sequence
  Synthetic construct
LNPAQSAEDVAAVAKMKQYEAVRSDEAVYGKGAKRAVPDEPVLAEHLVRKAAAIWALEEPFIDELRENEQDELFTDLEQP

SEQ ID NO: 66 - protein, 80 aa, Artificial Sequence
  Synthetic construct
LNPAQDAGDVAAVAKMKQYEAVRSDEAVYGKGAKRALPDEPTLAEHLVRKAAAIWALEEPFLDELRENEQDELLTELEQP

SEQ ID NO: 67 - protein, 80 aa, Artificial Sequence
  Synthetic construct
LNPAQSAGDVAAVAKMKQYEAVRSDEAVYGKGAKRAVPDEPTLAEHLVRKAAAIWALEEPFIDELRENEQDELFTELEMP

SEQ ID NO: 68 - protein, 80 aa, Artificial Sequence
  Synthetic construct
LNPAQDAGDVAAVAKMKQYEAVRPDEAVYGKGAKRSLPDEPTLAEHLVRKAAAIWALERPFLDELRRNEQDRLLIKLEQP

SEQ ID NO: 69 - protein, 80 aa, Artificial Sequence
  Synthetic construct
LNPAQDAGDVAAVAKMKQYEAVRSDEAVYGKGAKRSLPDEPTLAEHLVRKAAAIWALERPFLDELRRNEQDELLTKLEQP

SEQ ID NO: 70 - protein, 80 aa, Artificial Sequence
  Synthetic construct
LNPAQDAGDVAAVAKMKQYEAVRSDEAVYGKGAKRAVPDEPVLAEHLVRKAAAIWALERPFLDELRNNEQDELLTELEQP

SEQ ID NO: 71 - protein, 80 aa, Artificial Sequence
  Synthetic construct
LNPAQDAGDVAAVAKMKQYEAVRSDEAVYGKGAKRSVPDEPTLAEHLVRKAAAIWALEQPFLDELRRNEQDRLLTKLEQP

SEQ ID NO: 72 - protein, 80 aa, Artificial Sequence
  Synthetic construct
LNPAQSAGDVAAVAKMKQYEAVRSDEAVYGKGAKRAVPDEPTLAEHLVRKAAAIWALEQPFLDELRENEQDELLTKLEQP

SEQ ID NO: 73 - protein, 80 aa, Artificial Sequence
  Synthetic construct
LAAILAEMEFTGVNVDTKRLEQMGSELAEQLRAIEQRIYELAGQEFNINSPKQLGVILFEKLQLPVLKKTKTGYSTSADV

SEQ ID NO: 74 - protein, 80 aa, Artificial Sequence
  Synthetic construct
LATILAEMEFTGVKVDTKRLEQMGSELAEQLRAVEQRIYELAGQEFNINSPKQLGVILFEKLQLPVLKKTKTGYSTSADV

SEQ ID NO: 75 - protein, 80 aa, Artificial Sequence
  Synthetic construct
LATILAEMEFTGVKVDTKRLEQMGEELAEQLGAVEQRIYELAGQEFNINSPKQLGVILFEKLQLPVLKKTKTGYSTSADV

SEQ ID NO: 76 - protein, 80 aa, Artificial Sequence
  Synthetic construct
LILILAEMEFTGVDVDTKRLEQMGLELAEQLVEQEQRIYELAGQEFNINSPKQLGLILFEKLQLPVLKKTKTGYSTSADV

SEQ ID NO: 77 - protein, 80 aa, Artificial Sequence
  Synthetic construct
LSLILAEMEFTGVDVDTKRLEQMGLELAEQLGAQEQRIYELAGQEFNINSPKQLGVILFEKLQLPVLKKTKTGYSTSADV

SEQ ID NO: 78 - protein, 78 aa, Artificial Sequence
  Synthetic construct
LALILAEMEFTGVKVDTKRLEQMGEELAEQLKEVEQRIYELAGQEFNINSPKQLGVILFEKLQLPVLKKTKTGYSTSA

SEQ ID NO: 79 - protein, 80 aa, Artificial Sequence
  Synthetic construct
LILILAEMEFTGVDVDTKRLEQMGSELAEQLGAIEQRIYELAGQEFNINSPKQLGLILFEKLQLPVLKKTKTGYSTSADV

SEQ ID NO: 80 - protein, 80 aa, Artificial Sequence
  Synthetic construct
LAAILAEMEFTGVKVDTKRLEQMGEELAEQLKEVEQRIYELAGQEFNINSPKQLGVILFEKLQLPVLKKTKTGYSTSADV

SEQ ID NO: 81 - protein, 80 aa, Artificial Sequence
  Synthetic construct
LILILAEMEFTGVDVDTKRLEQMGLELAEQLVAQEQRIYELAGQEFNINSPKQLGVILFEKLQLPVLKKTKTGYSTSADV

SEQ ID NO: 82 - protein, 80 aa, Artificial Sequence
  Synthetic construct
LSLILAEMEFTGVDVDTKRLEQMGLELAEQLVEIEQRIYELAGQEFNINSPKQLGLILFEKLQLPVLKKTKTGYSTSADV

SEQ ID NO: 83 - protein, 80 aa, Artificial Sequence
  Synthetic construct
LALILAEMEFTGVKVDTKRLEQMGEELAEQLKAIEQRIYELAGQEFNINSPKQLGVILFEKLQLPVLKKTKTGYSTSADV

SEQ ID NO: 84 - protein, 80 aa, Artificial Sequence
  Synthetic construct
LALILAEMEFTGVKVDTKRLEQMGEELAEQLKEIEQRIYELAGQEFNINSPKQLGVILFEKLQLPVLKKTKTGYSTSADV

SEQ ID NO: 85 - protein, 80 aa, Artificial Sequence
  Synthetic construct
LALILAEMEFTGVKVDTKRLEQMGEELAEQLKAVEQRIYELAGQEFNINSPKQLGVILFEKLQLPVLKKTKTGYSTSADV

SEQ ID NO: 86 - protein, 80 aa, Artificial Sequence; identical to SEQ ID NO: 84
  Synthetic construct

SEQ ID NO: 87 - protein, 80 aa, Artificial Sequence
  Synthetic construct
LSSILAEMEFTGVKVDTKRLEQMGEELAEQLRAVEQRIYELAGQEFNINSPKQLGVILFEKLQLPVLKKTKTGYSTSADV

SEQ ID NO: 88 - protein, 80 aa, Artificial Sequence
  Synthetic construct
LEKLAPHHEIVENILHYRQLGKLQSTYIEGLLKVVRPDTGKVHTMFNQALTQTGRLSSAEPNLQNIPIRLEEGRKIRQAF

SEQ ID NO: 89 - protein, 80 aa, Artificial Sequence
  Synthetic construct
LEKLAPHHEIVENILHYRQLGKLQSTYIEGLLKVVHPDTGKVHTRFNQALTQTGRLSSTDPNLQNIPIRLEEGRKIRQAF

SEQ ID NO: 90 - protein, 80 aa, Artificial Sequence
  Synthetic construct
LEKLAPEHEIVENILHYRQLGKLQSTYIEGLLKVVRTDTGKVHTIFNQALTQTGRLSSAEPNLQNIPIRLEEGRKIRQAF

SEQ ID NO: 91 - protein, 80 aa, Artificial Sequence
  Synthetic construct
LEKLAPEHEIVENILHYRQLGKLQSTYIEGLLKVVRTDTGKVHTMFNQALTQTGRLSSADPNLQNIPIRLEEGRKIRQAF

SEQ ID NO: 92 - protein, 80 aa, Artificial Sequence
  Synthetic construct
LEKLAPHHEIVENILHYRQLGKLQSTYIEGLLKVVHPDTGKVHTMFNQALTQTGRLSSTEPNLQNIPIRLEEGRKIRQAF

SEQ ID NO: 93 - protein, 80 aa, Artificial Sequence
  Synthetic construct
LEKLAPHHEIVENILHYRQLGKLQSTYIEGLLKVVRPDTGKVHTMFNQALTQTGRLSSTEPNLQNIPIRLEEGRKIRQAF

SEQ ID NO: 94 - protein, 80 aa, Artificial Sequence
  Synthetic construct
LEKLAPEHEIVENILHYRQLGKLQSTYIEGLLKVVDTDTGKVHTMFNQALTQTGRLSSAEPNLQNIPIRLEEGRKIRQAF

SEQ ID NO: 95 - protein, 80 aa, Artificial Sequence
  Synthetic construct
LEKLAPHHEIVENILHYRQLGKLQSTYIEGLLKVVHPDTGKVHTIFNQALTQTGRLSSTEPNLQNIPIRLEEGRKIRQAF

SEQ ID NO: 96 - protein, 80 aa, Artificial Sequence
  Synthetic construct
LEKLAPHHEIVENILHYRQLGKLQSTYIEGLLKVVHPDTGKVHTMFNQALTQTGRLSSAEPNLQNIPIRLEEGRKIRQAF

SEQ ID NO: 97 - protein, 80 aa, Artificial Sequence
  Synthetic construct
LEKLAPHHEIVENILHYRQLGKLQSTYIEGLLKVVDTDTGKVHTMFNQALTQTGRLSSAEPNLQNIPIRLEEGRKIRQAF

SEQ ID NO: 98 - protein, 80 aa, Artificial Sequence
  Synthetic construct
LEKLAPHHEIVENILHYRQLGKLQSTYIEGLLKVVHPDTGKVHTRFNQALTQTGRLSSTEPNLQNIPIRLEEGRKIRQAF

SEQ ID NO: 99 - protein, 80 aa, Artificial Sequence
  Synthetic construct
LEKLAPEHEIVENILHYRQLGKLQSTYIEGLLKVVRTDTGKVHTMFNQALTQTGRLSSAEPNLQNIPIRLEEGRKIRQAF

SEQ ID NO: 100 - protein, 80 aa, Artificial Sequence
  Synthetic construct
VPSEPDWLIFAADYSQIELRVLAHIADDDNLIEAFQRDLDIHTKTAMDIFHVSEEEVTANMRRQAKAVNFGIVYGISDYG

SEQ ID NO: 101 - protein, 80 aa, Artificial Sequence
  Synthetic construct
VPSEPLWLIFAADYSQIELRVLAHIADDDNLAEAFQRDLDIHTKTAMDIFHVSEEEVTSRMRRQAKAVNFGIVYGISDYG

SEQ ID NO: 102 - protein, 80 aa, Artificial Sequence
  Synthetic construct
VPSEPLWLIFAADYSQIELRVLAHIADDDNLAEAFRRDLDIHTKTAMDIFHVSEEEVTARMRRQAKAVNFGIVYGISDYG

SEQ ID NO: 103 - protein, 80 aa, Artificial Sequence
  Synthetic construct
VPSEPDWLIFAADYSQIELRVLAHIANDENLIEAFRRDLDIHTKTAMDIFHVSEDEVTANMRRQAKAVNFGIVYGISDYG

SEQ ID NO: 104 - protein, 80 aa, Artificial Sequence
  Synthetic construct
VPSEPDWLIFAADYSQIELRVLAHIANDDNLIEAFRRDMDIHTKTAMDVFHVSEDEVTSNMRRQAKAVNFGIVYGISDYG

SEQ ID NO: 105 - protein, 80 aa, Artificial Sequence
  Synthetic construct
VPSEPLWLIFAADYSQIELRVLAHIANDDNLAEAFQRDMDIHTKTAMDVFHVSEEEVTARMRRQAKAVNFGIVYGISDYG

SEQ ID NO: 106 - protein, 80 aa, Artificial Sequence
  Synthetic construct
VPSEPDWLIFAADYSQIELRVLAHIADDDNLIEAFRRDLDIHTKTAMDIFHVSEEEVTANMRRQAKAVNFGIVYGISDYG

SEQ ID NO: 107 - protein, 80 aa, Artificial Sequence
  Synthetic construct
VPSEPDWLIFAADYSQIELRVLAHIADDDNLIEAFRRDLDIHTKTAMDIFHVSEDEVTANMRRQAKAVNFGIVYGISDYG

SEQ ID NO: 108 - protein, 80 aa, Artificial Sequence
  Synthetic construct
VPSEPDWLIFAADYSQIELRVLAHIANDDNLIEAFRRDLDIHTKTAMDIFHVSEDEVTANMRRQAKAVNFGIVYGISDYG

SEQ ID NO: 109 - protein, 80 aa, Artificial Sequence
  Synthetic construct
VPSEPLWLIFAADYSQIELRVLAHIANDENLAEAFQRDLDIHTKTAMDVFHVSEEEVTSRMRRQAKAVNFGIVYGISDYG

SEQ ID NO: 110 - protein, 80 aa, Artificial Sequence
  Synthetic construct
VPSEPDWLIFAADYSQIELRVLAHIADDENLIEAFRRDLDIHTKTAMDIFHVSEEEVTANMRRQAKAVNFGIVYGISDYG

SEQ ID NO: 111 - protein, 80 aa, Artificial Sequence; identical to SEQ ID NO: 106
  Synthetic construct

SEQ ID NO: 112 - protein, 80 aa, Artificial Sequence
  Synthetic construct
LAQNLNITRKEAAEFIERYFASFPGVKQYMENIVQEAKQKGYVTTLLHRRRYLPDITSRNFNVRSFAERTAMNTPIQGSA

SEQ ID NO: 113 - protein, 80 aa, Artificial Sequence
  Synthetic construct
LSQNLNITRKEAAEFIERYFESFPGVKRYMENIVQEAKQKGYVTTLLHRRRYLPDITSRNFNVRSFAERTAMNTPIQGSA

SEQ ID NO: 114 - protein, 80 aa, Artificial Sequence
  Synthetic construct
LAQNLNITRKEAAEFIERYFASFPGVKRYMEDIVQEAKQKGYVTTLLHRRRYLPDITSRNFNVRSFAERTAMNTPIQGSA

SEQ ID NO: 115 - protein, 80 aa, Artificial Sequence
  Synthetic construct
LAQNLNITRKEAAEFIERYFESFPGVKQYMENIVQEAKQKGYVTTLLHRRRYLPDITSRNFNVRSFAERTAMNTPIQGSA

SEQ ID NO: 116 - protein, 80 aa, Artificial Sequence
  Synthetic construct
LAQNLNIKRKEAAEFIERYFASFPGVKRYMEVIVQEAKQKGYVTTLLHRRRYDPDITSRNFNVRSFAERMAMNTPIQGSA

SEQ ID NO: 117 - protein, 80 aa, Artificial Sequence
  Synthetic construct
LSQNLNITRKEAAEFIERYFESFPGVKQYMEDIVQEAKQKGYVTTLLHRRRYLPDITSRNFNVRSFAERTAMNTPIQGSA

SEQ ID NO: 118 - protein, 80 aa, Artificial Sequence
  Synthetic construct
LAQNLGITRKEAAEFIERYFASFPGVKQYMEDIVQEAKQKGYVTTLLHRRRYLPEITSRNFNLRSFAERTAMNTPIQGSA

SEQ ID NO: 119 - protein, 80 aa, Artificial Sequence
  Synthetic construct
LAQNLGITRKEAAEFIERYFASFPGVKEYMEEIVQEAKQKGYVTTLLHRRRYLPDITSRNFNVRSFAERMAMNTPIQGSA

SEQ ID NO: 120 - protein, 80 aa, Artificial Sequence
  Synthetic construct
LAQNLNITRKEAAEFIERYFASFPGVKRYMENIVQEAKQKGYVTTLLHRRRYLPDITSRNFNVRSFAERTAMNTPIQGSA

SEQ ID NO: 121 - protein, 80 aa, Artificial Sequence
  Synthetic construct
LAQNLNITRKEAAEFIERYFESFPGVKRYMENIVQEAKQKGYVTTLLHRRRYLPDITSRNFNVRSFAERTAMNTPIQGSA

SEQ ID NO: 122 - protein, 80 aa, Artificial Sequence
  Synthetic construct
LSQNLNITRKEAAEFIERYFASFPGVKQYMENIVQEAKQKGYVTTLLHRRRYLPDITSRNFNVRSFAERTAMNTPIQGSA

SEQ ID NO: 123 - protein, 80 aa, Artificial Sequence
  Synthetic construct
LAQNLNIKRKEAAEFIERYFASFPGVKQYMEVIVQEAKQKGYVTTLLHRRRYDPDITSRNFNVRSFAERMAMNTPIQGSA

SEQ ID NO: 124 - protein, 80 aa, Artificial Sequence
  Synthetic construct
LSQNLGITRKEAAEFIERYFESFPGVKEYMEDIVQEAKQKGYVTTLLHRRRYLPEITSRNFNLRSFAERTAMNTPIQGSA

SEQ ID NO: 125 - protein, 80 aa, Artificial Sequence
  Synthetic construct
LSQNLNITRKEAAEFIERYFASFPGVKRYMEEIVQEAKQKGYVTTLLHRRRYLPDITSRNFNVRSFAERTAMNTPIQGSA

SEQ ID NO: 126 - protein, 76 aa, Artificial Sequence
  Synthetic construct
ADIIKKAMIDLAARLKEEQLQARLLLQVHDELILEAPKEEIERLCELVPEVMEQAVTLRVPLKVDYHYGPTWYDAK

SEQ ID NO: 127 - protein, 76 aa, Artificial Sequence
  Synthetic construct
ADIIKKAMIDLAARLKEERLQARLLLQVHDELILEAPKEEIERLCRLVPEVMEQAVTLRVPLKVDYHYGPTWYDAK

SEQ ID NO: 128 - protein, 76 aa, Artificial Sequence
  Synthetic construct
ADIIKKAMIDLAARLKEERLQARLLLQVHDELILEAPKEEIERLCKLVPEVMENAVTLRVPLKVDYHYGPTWYDAK

SEQ ID NO: 129 - protein, 76 aa, Artificial Sequence
  Synthetic construct
ADIIKKAMIDLAARLKEEQLQARLLLQVHDELILEAPKEEMERLCVLVPEVMEQAVRLRVPLKVDYHYGWTWYDAK

SEQ ID NO: 130 - protein, 76 aa, Artificial Sequence
  Synthetic construct
ADIIKKAMIDLAARLKEERLQARLLLQVHDELILEAPKEEMERLCRLVPEVMEQAVTLRVPLKVDYHYGPTWYDAK

SEQ ID NO: 131 - protein, 76 aa, Artificial Sequence
  Synthetic construct
ADIIKKAMIDMAARLKEEQLQARLLLQVHDELILEAPKEEMERLEELVPEVMEHAVTLRVPLKVDYHYGWTWYDAK

SEQ ID NO: 132 - protein, 76 aa, Artificial Sequence
  Synthetic construct
ADIIKKAMIDMAARLKEEQLQARLLLQVHDELIFEAPKEEIERLEELVPEVMENAVTLRVPLKVDYHYGWTWYDAK

SEQ ID NO: 133 - protein, 76 aa, Artificial Sequence
  Synthetic construct
ADIIKKAMIDLAARLKEERLQARLLLQVHDELILEAPKEEIERLCQLVPEVMEQAVELRVPLKVDYHYGPTWYDAK

SEQ ID NO: 134 - protein, 76 aa, Artificial Sequence
  Synthetic construct
ADIIKKAMIDMAARLKEERLQARLLLQVHDELIFEAPKEEIERLEKLVPEVMEHAVELRVPLKVDYHYGPTWYDAK

SEQ ID NO: 135 - protein, 76 aa, Artificial Sequence
  Synthetic construct
ADIIKKAMIDLAARLKEEQLQARLLLQVHDELIFEAPKEEMERLEELVPEVMEHAVTLRVPLKVDYHYGWTWYDAK

SEQ ID NO: 136 - protein, 76 aa, Artificial Sequence
  Synthetic construct
ADIIKKAMIDMAARLKEEQLQARLLLQVHDELIFEAPKEEIERLCELVPEVMENAVTLRVPLKVDYHYGWTWYDAK

SEQ ID NO: 137 - protein, 76 aa, Artificial Sequence
  Synthetic construct
ADIIKKAMIDLAARLKEERLQARLLLQVHDELILEAPKEEIERLCQLVPEVMEQAVTLRVPLKVDYHYGPTWYDAK

SEQ ID NO: 138 - protein, 76 aa, Artificial Sequence
  Synthetic construct
ADIIKKAMIDLAARLKEERLQARLLLQVHDELILEAPKEEIERLCKLVPEVMEQAVELRVPLKVDYHYGPTWYDAK

SEQ ID NO: 139 - DNA, 46 bp, Artificial Sequence
  Synthetic construct
cagccagccgcagcacgttcgctcataggagatatggtagagccgc

SEQ ID NO: 140 - DNA, 51 bp, Artificial Sequence
  Synthetic construct
gagagaatttgtaccacctcccaccgggcacatagcagtcctagggacagt

SEQ ID NO: 141 - DNA, 22 bp, Artificial Sequence
  Synthetic construct
ggcttggctctgctaacacgtt

SEQ ID NO: 142 - DNA, 22 bp, Artificial Sequence
  Synthetic construct
ggacgtttgtaatgtccgctcc

SEQ ID NO: 143 - DNA, 17 bp, Artificial Sequence
  Synthetic construct
ctgcatacgacgtgtct

SEQ ID NO: 144 - DNA, 20 bp, Artificial Sequence
  Synthetic construct
accatctatgactgtacgcc

SEQ ID NO: 145 - DNA, 24 bp, Artificial Sequence
  Synthetic construct
cgccagggttttcccagtcacgac

SEQ ID NO: 146 - DNA, 20 bp, Artificial Sequence
  Synthetic construct
agaacgggaagcttgtcatc

SEQ ID NO: 147 - DNA, 18 bp, Artificial Sequence
  Synthetic construct
cgaacatgggggcatcag